(19) United States
(12) Patent Application Publication
(10) Pub. No.: US 2011/0111434 A1
(43) Pub. Date: May 12, 2011

(54) COLON STEM CELLS ASSOCIATED WITH COLITISAND COLORECTAL CANCER AND METHODS OF USE

(76) Inventors: Emina H. Huang, Gainsville, FL (US); Edward W. Scott, Gainesville, FL (US); Joseph E. Carpentino, Gainesville, FL (US)

(21) Appl. No.: 13/003,888

(22) PCT Filed: Jul. 14, 2009
(86) PCT No.: PCT/US2009/050534

§ 371 Date: Jan. 13, 2011

Related U.S. Application Data
(60) Provisional application No. 61/080,813, filed on Jul. 15, 2008.

(51) Int. Cl. G01N 33/566; C12N 5/071; C12Q 1/02; C12N 5/09

(52) U.S. Cl. 435/7.21; 435/325; 435/347; 435/29; 435/375

(57) ABSTRACT

The disclosure provides methods of isolating and propagating self-renewing colonic stem/progenitor cells (CS/PCs) that express aldehyde dehydrogenase (ALDH1), from colon cancer and colitis tissues, as well as from normal colon tissue, methods of identifying agents for modulating the proliferative status of such cells, an methods of screening patients having colitis for an increased risk of colorectal cancer. Novel methods of adherent cell culture propagation of CS/PC involving use of colon-specific fibroblastic stromal cells (CFSt) {i.e. the “niche” cells) as support cells (e.g., “feeder cells”). The present disclosure encompasses an isolated mammalian pluripotent colon epithelial stem/progenitor cell (CS/PC), or a population of said cells, where each CS/PC may comprise a detectable marker, where the detectable marker is aldehyde dehydrogenase 1 (ALDH1), and where the isolated population of mammalian pluripotent CS/PCs is substantially free of cells that do not have the detectable ALDH 1 marker. The disclosure encompasses further provides methods for determining the prognosis for a patient for developing a colon cancer, the method detecting the presence of at least one marker in a tissue section from a patient, the marker or plurality of markers indicating the presence of a pluripotent colon epithelial stem/progenitor cells and indicating the prognosis of the patient for developing a colon cancer.

### CROSS-REFERENCE TO RELATED APPLICATIONS

This application claims priority to U.S. Provisional Patent Application Ser. No. 61/080,813, entitled “COLON EPITHELIAL STEM CELLS ASSOCIATED WITH COLITIS AND COLORECTAL CANCER AND METHODS OF USE” filed on Jul. 15, 2008, the entirety of which is hereby incorporated by reference.

### STATEMENT AS TO FEDERALLY-SPONSORED RESEARCH

This disclosure was made with U.S. government support under grant number HL70738, HL 75258, and NIH T32 DK 074367 awarded by the National Institutes of Health. The U.S. government has certain rights in the disclosure.

### TECHNICAL FIELD

The present disclosure is generally related to the isolation, culturing, and characterization of colon epithelial stem/progenitor cells and colonic fibroblast stromal cells associated with colorectal cancer and colitis, and uses thereof as predictors of tumorigenicity in a patient.

### BACKGROUND

Roughly 153,000 people are diagnosed with colorectal cancer (CRC) annually, making it the third most common form of cancer in the United States. In most cases, the cancer is preceded by the occurrence of small, mushroom-shaped bumps (polyps), which can be identified with proper monitoring and removed. Inflammatory bowel disease, or IBD, includes ulcerative colitis and Crohn's disease, and affects 4-6 per 100,000 people in North America alone. Patients with long-standing inflammatory bowel disease have up to a 20-fold increased risk of colon cancer. Colitis-associated cancer is a devastating example of inflammation-associated cancer. Despite the frequency of these diseases, underlying mechanisms responsible for the progression from colitis to cancer are poorly understood.

### SUMMARY

The disclosure provides methods of isolating and propagating self-renewing colonic stem/progenitor cells (CS/PCs) that express aldehyde dehydrogenase (ALDH1), from colon cancer and colitis tissues, as well as from normal colon tissue, methods of identifying agents for modulating the proliferative status of such cells, and methods of screening patients having colitis for an increased risk of colorectal cancer Novel methods of adherent cell culture propagation of CS/PC involving use of colon-specific fibroblastic stromal cells (CFSt) (i.e. the “niche” cells) as support cells (e.g., “feeder cells”) are also described herein.

The studies described herein resulted in the discoveries that ALDH1-positive CS/PCs are present in increased numbers in colitic epithelium and colon cancer as compared to normal colon tissue. Furthermore, a subpopulation of individuals suffering from colitis is more likely to develop colorectal cancer and that this increased risk can be screened by analyzing ALDH1-positive CS/PC levels.

Even in the absence of documented dysplasia or cancer, colitic epithelium has the capacity to foster tumorigenic growth. The present disclosure provides evidence that colitis-associated fibroblasts stimulate proliferation of CS/PCs, resulting in methods of adherent cell culture propagation of CS/PCs involving use of colon-specific fibroblastic stromal cells (CFStcs) (i.e the “niche” cells) as support cells (e.g., “feeder cells”) required for proper maintenance, propagation, and differentiation of CS/PCs. The CFStcs described herein, when cultured, act as support or feeder cells to CS/PCs and provide a culture environment that resembles the colon microenvironment, thus maintaining CS/PCs in a more stem-like condition. The ALDH1-positive CS/PCs described herein were shown to be capable of forming suspended spherical clonal colonies, or “colon spheres”, in anchorage independent serum starved conditions.

The studies described herein show that another marker, aldehyde dehydrogenase 1 (ALDH1), is useful for isolating populations enriched for CS/PC, but can also be used to predict patient outcome in those suffering from various forms of colitis and/or cancer. This marker is particularly useful because it labels the inactive or slowly multiplying stem cells, allowing more rigorous detection of these cells.

One aspect of the present disclosure encompasses an isolated mammalian pluripotent colon epithelial stem/progenitor cell (CS/PC), or a population of said cells, where each CS/PC may comprise a detectable marker, where the detectable marker is aldehyde dehydrogenase 1 (ALDH1), and where the isolated population of mammalian pluripotent CS/PCs is substantially free of cells that do not have the detectable ALDH1 marker.

In embodiments of this aspect of the disclosure, the isolated mammalian pluripotent colon epithelial stem/progenitor cell (CS/PC), or population of said cells, can be isolated from a normal colon, a cancerous colon, or a colitic colon.

Another aspect of the present disclosure provides embodiments of a population of cultures mammalian pluripotent colon epithelial stem/progenitor cells (CS/PCs) according to any of the above embodiments, where the population of CS/PCs may be cultured under in vitro conditions, thereby forming a cell-based spheroid.

Yet another aspect of the present disclosure provides for a fibroblast-epithelial cell co-culture comprising an isolated mammalian pluripotent colon epithelial stem/progenitor cell (CS/PC), or population of said cells, and an isolated mammalian colonic stromal fibroblast cell (CS/PC), or population of said cells.

Still another aspect of the present disclosure encompasses methods of isolating a mammalian pluripotent colon epithelial stem/progenitor cell (CS/PC), or a population of said cells, from an animal or human colon, where each CS/PC comprises a detectable marker, where the detectable marker is aldehyde dehydrogenase 1 (ALDH1), said method comprising the steps of: (i) obtaining a tissue sample from the colon of an animal or human subject; (ii) disrupting the tissue sample, thereby obtaining a cell suspension, where the cell suspension is substantially comprised of single cells; (iii) contacting the cell suspension with at least one ligand species capable of selectively binding to a cell marker, where each cell marker, or combination of markers selectively identifies a mammalian pluripotent colon epithelial stem/progenitor cell (CS/PC), and where each ligand species comprises a detectable label; (iv) identifying a cell or population of cells binding to the ligand species or combination of ligand species; and (v) isolating the identified cell or population of cells selectively binding to the ligand species or combination of ligand species from a population of cells not selectively binding to the ligand species or combination of ligand species, thereby obtaining an isolated population of mammalian pluripotent colon epithelial stem/progenitor (CS/PC) cells.

Yet another aspect of the present disclosure encompasses methods for producing self-renewing pluripotent CS/PC clones comprising the steps of: culturing colonspheres; dissociating colonspheres into single cells; culturing said single cells to near confluence and harvesting; reseeding harvested cells into suspension (non-adherent) cultures; identifying self-renewing cells by formation of secondary spheres; and isolating self-renewing pluripotent CS/PC clones.

Another aspect of the disclosure encompasses methods for determining the prognosis for a patient for developing a colon cancer, the method comprising: (i) obtaining an isolated tissue sample from the colon of an animal or human patient; (ii) obtaining a tissue section from the isolated tissue section; and (iii) detecting the presence of at least one marker in the tissue section, where the marker or plurality of markers indicate the presence of a pluripotent colon epithelial stem/progenitor cell (CS/PC), thereby indicating the prognosis of the patient for developing a colon cancer.

Still yet another aspect of the present disclosure encompasses kits comprising a container comprising a detectable ligand, or a combination of detectable ligands, where each species of detectable ligand specifically binds to an individual marker, where a marker alone, or in combination with at least one other marker, identifies a colonic stem/progenitor cell from a population of cells of a mammal, and instructions for the use of the detectable ligand or combination of markers to identify a colonic stem/progenitor cell from a population of cells of a mammal.

Another aspect of the disclosure is a method of identifying a candidate therapeutic compound effective in reducing the proliferative status of a colonic stem/progenitor cell of a mammal, comprising the steps of: culturing a population, or plurality of populations, of isolated CS/PCs, with a plurality of candidate therapeutic agents, and identifying a candidate therapeutic agent that reduces the proliferation status of a culture of a population of CS/PCs.

Another aspect of the present disclosure provides methods of modulating the proliferative status of a colonic cancer cell comprising: contacting a colonic cell with an effective amount of an agent, where the agent selectively binds to a cell surface marker, and where the agent when bound to the cell surface marker inhibits binding of a factor to the cell, thereby modulating the proliferative status of the colonic cell.

ALDH1-positive epithelial cells can be isolated from a patient using biopsy techniques as well less invasive means, such as isolation from the feces, rectal mucus, or blood. The cells, methods and kits described herein allow for earlier detection and quantification of pre-cancerous and metastatic cells and will allow the clinician to prescribe more effective treatments for patients with colitis, or early stage cancer.

### BRIEF DESCRIPTION OF THE DRAWINGS

The disclosure is pointed out with particularity in the appended claims. The disclosure may be better understood by referring to the description taken in conjunction with the accompanying drawings, in which:

FIG. 1, panels A-F, shows a series of photomicrographs of normal colon cells and cancer colon cells subjected to immunohistochemistry for the detection of the colonic epithelial stem cell markers ALDH1, CD44, and CD133.

FIGS. 2A-2F show data that flow cytometry from primary human colorectal cancer for ALDH1 may be used to isolate CRC-initiating cells.

FIG. 2A shows a digital photomicrograph of a hematoxylin-eosin (H&E)-stained primary human colorectal cancer (CRC). 200×.

FIG. 2B shows a flow cytometry histogram of ALDH1-positive cells. To verify that ALDH1 could be utilized to select colon cancer cells with tumorigenic potential, the ALDEFLUOR™ (Stem Cell Technologies, Vancouver, BC) system was used, and cells were sorted from primary CRC isolates. ALDH1^{high} cells were 0.9% of the gated population.

FIG. 2C is a digital image of a NOD-SCID mouse injected with sorted cell populations. Mice were injected in the right flank with ALDH1^{high}/ESA^{high} cells. The marker endothelial-specific antigen (ESA) was included to limit the ALDH1^{high} population to epithelial cells. In the opposite flank, the control population of injected cells was an equal number of Aldefluor/ESA^{high} cells. Within 4-8 weeks of injection, visible and palpable tumors were detectable, but were not apparent even when cells from normal tissues were implanted (normal tissue colon isolates=10).

FIG. 2D shows a digital photomicrograph of an H&E-stained xenograft revealing an adenocarcinoma invading the fascia, and which resembled the parental adenocarcinoma.

FIG. 2E shows a flow cytometry histogram showing that cells positive for ALDH1 (0.9%) are maintained in resulting xenografts. Further passages using these markers demonstrated heterogeneity in distribution of Aldefluor^{high}, thus preserving the relative distribution of Aldefluor^{high} established in the primary lesion.

FIG. 2F is a graph showing that growth latency was dependent on the number of injected cells.

FIGS. 3A-3D is a series of digital photomicrographs of colon cells subjected to immunohistochemistry for ALDH1. FIG. 3A: Normal colonic epithelium revealing expression of ALDH1-positive cells at the base of the crypt (arrow). FIG. 3B: Invasive colon cancer demonstrating numerous ALDH1-positive tumor cells at the invasive leading front. FIG. 3C: In colitis, the expression of ALDH1-positive cells was expanded. FIG. 3D: In colitis-associated cancer, ALDH1 expression was seen throughout. (Images A, B 200×; C, D 400×; N=3).

FIGS. 4A-4D provides a flow chart incorporating a series of digital photomicrographs and photographs showing the retrieval and engraftment of colitic tissue. FIG. 4A: H&E stained 2 mm of colon tissue (40×) implanted into the flank of a NOD-SCID mouse. The mice were observed for xenograft incorporation and growth. FIG. 4B: when the tumor had grown sufficiently (arrow), the mass was removed and the cells were dissociated, subjected to flow cytometry, and implanted. FIG. 4C: as few as 200 cells were capable of reconstituting a tumor mass. FIG. 4D: H&E staining of a tumor reveals anaplastic cells (400×; N=3).

FIG. 5 is a graph illustrating cancer-associated fibroblasts isolated and cultured in vitro.

FIGS. 6A and 6B show a series of digital images of cytokine arrays for the detection of colon cancer and colitic-associated fibroblast markers (and tables mapping the positions of the array spots are shown below each series of assays.). The Panomics system was used to assay culture supernatants.

FIG. 6A shows arrays A: Normal colon fibroblast line (ATCC CRL 1541); B: Colon cancer fibroblast line (ATCC CRL 7213); C: Serum-free media (negative control); and D: Colitis stromal fibroblasts (primary isolate).

FIG. 6B shows the results with the Panomics angiogenesis array used to assay culture supernatants. Array E: Normal colon (primary isolate); Array F: Colon cancer (primary isolate); G: Colitis.

FIGS. 7A-7K show a series of digital photomicrographs of spheroid cultures from colon cancer and colitis, a schematic of a promoter, and digital phase contrast photomicrographs of spheroid cultures. FIGS. 7A-7E: Representative digital micrographs from primary, secondary, tertiary, pentary and septary generations, respectively (line=100 microns, 400×). FIG. 7F: H&E-stained spheroid culture suspended in agarose (400×). FIG. 7G: Spheroid culture obtained from ALDH1-enrichment of dissociated colon cancer. Image H: Spheroid culture of ALDH1-enrichment of dissociated cells from colitis. FIG. 71: Schematic of CMV-driven promoter for dsRed in a lentiviral construct. FIG. 7J: Phase contrast of spheroid culture post-transduction with dsRed lentiviral construct. FIG. 7K: ds Red-expressing spheroid cultures. 72 hours post transduction, dsRed-expressing sphere cultures are visible.

FIGS. 8A-8D shows a digital photograph, a graph, and a pair of photomicrographs demonstrating the establishment of co-cultures of spheres and cancer-associated fibroblasts in vivo.

FIG. 8A is a digital image showing tumor masses result from co-injection of colon spheres and colon cancer-associated fibroblasts (CAF).

FIG. 8B is a graph showing the tumor latency of injected cells.

FIG. 8C is a digital photomicrograph of an H&E-stained colon sphere (1000 cells) with cancer associated fibroblasts (1000 cells, N=2, 400×).

FIG. 8D is a digital photomicrograph of an H&E-stained colon sphere alone (2000 dissociated cells, N=1, 400×).

FIGS. 9A-9D illustrate the immunohistochemistry of ALDH1 expression in normal colon, colitis, and colon cancer.

FIG. 9A is a digital photomicrograph showing normal colonic epithelium revealing expression of ALDH1 at the cells at and near the base of the crypt (arrows) with several cells found further up the crypt (arrowheads).

FIG. 9B is a digital photomicrograph showing the colitic milieu demonstrating expansion of ALDH1 staining at the base of the crypt compared to baseline normal mucosa.

FIG. 9C is a digital photomicrograph showing, in colitis-associated cancer, the expression of ALDH1 is greatly expanded (C).

FIG. 9D is a graph showing ALDH1-expressing cells in the epithelial portions of the primary tissue samples have an increase in immunoreactive ALDH1-expressing cells in the colitic colon compared to normal colon tissue.

FIGS. 10A and 10B are a series of digital photomicrograph images showing the generation of xenografts from colon cancer and colitis.

FIG. 10A shows where a portion of the colon was removed at operation and tissue was implanted into the flank of NOD-SCID mice. As few as 25 cells of either ESA^{high}CD44^{high} or ESA^{high}ALDH1^{high} were capable of reconstituting a tumor, the histology of which strongly resembled the parent tumor (N=10).

FIG. 10B shows where colitic colon tissue was implanted into the flank of NOD-SCID mice and observed for xenograft incorporation and growth. As few as 50 ESA^{high}ALDH1^{high} cells were capable of reconstituting a tumor, and H&E staining of tumor revealed anaplastic cells (FIG. 10B, middle panel; N=3). With serial passaging, xenografts from colitic tissues revealed an adenocarcinoma, with the epithelium forming gland-like structures (FIG. 10B, right panel; N=3). Scale Bar=200 μm.

FIGS. 11A-11C show a series of digital photomicrograph images illustrating a marker analysis of primary tissue, xenografts, and spheres from malignant colon tissue. Expression of CD44 and ALDH1 was abundant in all tissue types examined (FIGS. 11A-11C, right and middle panels, respectively). MUC2 expression in cancerous colon tissue was found in some crypts (FIG. 11A, right panel), while others showed no staining (arrowheads). MUC2 expression was relatively low and sporadic in the xenografts and spheres (FIGS. 11B and 11C, right panels, respectively). Scale bar=200 μm.

FIGS. 12A-12C show a series of digital photomicrograph images illustrating a marker analysis of primary tissue, xenografts, and spheres from colitic colons. Expression of CD44 was abundant in all tissue types examined (FIGS. 12A-12C, left panels). ALDH1 expression was expanded from the base of the crypts of colitic colons (FIG. 12A, middle panel), were found to be rare in xenografted tissue (FIG. 12B, middle panel), and was found in clusters in colitis-spheres (FIG. 12C, middle panel). MUC2 expression was abundant in the colitic and xenograft tissue (A and B, right panels) but was not observed in colitis-spheres (FIG. 12C, right panel). Scale bar=200 μm.

FIG. 13A is a digital image showing a cytokine array analysis of conditioned media from normal (top), colitic (middle), and malignant (bottom) human colon. Cytokines IL-6 (arrowhead), and IL-8 (arrow) are relatively absent in normal conditions (N=4), with increases in colitic (N=3) and malignant conditions (N=3).

FIG. 13B shows digital images illustrating IL-8 receptor expression (CXCR1) in adenocarcinoma generated from colitic tissues revealing widespread expression (upper panel, N=2). IL-8 receptor expression (CXCR1) in adenocarcinoma originating from human sporadic adenocarcinoma (lower panel, N=4). CXCR1 is also widely expressed in xenografts (passage 7) from sporadic colon cancer and resulting tumorigenic growth (B, lower panel). Co-injection of normal, colitic, or cancer-associated fibroblasts reveal a role for stromal elements in tumorigenesis and corresponding cytokine arrays (C) demonstrate a likelihood that IL and/or IL-8 play a role p<0.0001.

FIG. 13C is a graph illustrating relative cytokine/chemokine expression for IL-6 (left) and IL-8 (right).

FIG. 13D is a graph illustrating that pellets containing antibodies to IL-8 or IL-6 resulted in significantly reduced tumor sizes, and resulted in persistent inhibition of tumorigenesis until the source of antibodies was exhausted at about 8 weeks post-implant (arrow). *p<0.03; **, p<0.01; N> or =4 per group. Scale bar=100

FIG. 14 shows a series of digital fluorescence photomicrographs illustrating ALDH1 and vimentin expression in xenografts and spheroid colonies.

Panels A-C: Colitis xenografts. Panel A: immunofluorescence stained (FITC) for ALDH1; Panel B: immunofluorescence stained (PE) for vimentin; Panel C: merged images shown in Panels A and B. Co-localization ALDH1 and vimentin expression in rare cells within the glandular structures are indicated by arrows.

Panels D-F: Colitis-derived spheroid colonies. Panel D: immunofluorescence stained (FITC) for ALDH1; Panel E: immunofluorescence stained (PE) for vimentin; Panel F: merged images shown in Panels A and B. Co-localization ALDH1 and vimentin expression in rare cells within the spheroid is indicated by arrows.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following abbreviations and definitions are provided and should be used unless otherwise indicated.

### Abbreviations

CS/PC(s), colon epithelial stem/progenitor cell(s); H&E, hematoxylin and eosin; ALDH1, aldehyde dehydrogenase; CRC, colorectal cancer; CFStc(s), colon-specific fibroblastic stromal cell(s); ESA, endothelial-specific antigen; FITC, fluorescein isothiocyanate; EMT, epithelial-mesenchymal transition.

### Description

The terms “suspension of cells” or “cells in suspension” as used herein refer to cells that do not adhere to a solid support.

The terms “primary culture” and “primary cells” refer to cells derived from intact or dissociated tissues or organ fragments. A culture is considered primary until it is passaged (or subcultured) after which it is termed a “cell line” or a “cell strain.” The term “cell line” does not imply homogeneity or the degree to which a culture has been characterized. A cell line is termed “clonal cell line” or “clone” if it is derived from a single cell of a population of cultured cells.

The term “cell engraftment” as used herein refers to the process by which cells such as, but not limited to, colonic epithelial stem cells, are delivered to, and become incorporated into, a recipient animal.

The terms “cell surface antigen” and “cell surface marker” as used herein may be any antigenic structure on the surface of a cell. The cell surface antigen may be, but is not limited to, a tumor associated antigen, a growth factor receptor, a viral-encoded surface-expressed antigen, an antigen encoded by an oncogene product, a surface epitope, a membrane protein which mediates a classical or atypical multi-drug resistance, an antigen which mediates a tumorigenic phenotype, an antigen which mediates a metastatic phenotype, an antigen which suppresses a tumorigenic phenotype, an antigen which suppresses a metastatic phenotype, an antigen which is recognized by a specific immunological effector cell such as a T-cell, and an antigen that is recognized by a non-specific immunological effector cell such as a macrophage cell or a natural killer cell. Examples of “cell surface antigens” include, but are not limited to, ALDH1, ESA, c-kit, Mud, Muc2, CK19, Oct 3/4, Nanog, activated Stat-3, CXCR4, CXCR1/2, CD133, SCA-1, Tra-1-60, CD44, CD73, CD90, CD105, vimentin, and Stro-1. Cell surface molecules may also include carbohydrates, proteins, lipoproteins or any other molecules or combinations thereof, that may be detected by selectively binding to a ligand or labeled molecule and by methods such as, but not limited to, flow cytometry.

The term “cell surface indicator” as used herein refers to a compound or a plurality of compounds that will bind to a cell surface antigen directly or indirectly and thereby selectively indicate the presence of the cell surface antigen. Suitable “cell surface indicators” include, but are not limited to, cell surface antigen-specific monoclonal or polyclonal antibodies, or derivatives or combinations thereof, and which may be directly or indirectly linked to a signaling moiety. The “cell surface indicator” may be a ligand that can bind to the cell surface antigen, wherein the ligand may be a protein, peptide, carbohydrate, lipid or nucleic acid that is directly or indirectly linked to a signaling moiety. It is contemplated that such a ligand may be labeled with such as a detectable label, or a tag that may allow a target cell binding to the ligand to be isolated from a population of cells not binding the ligand.

The term “directly delivering” as used herein refers to delivering a pharmaceutically acceptable agent or preparation, or a suspension of isolated cells, into a mass of target cells or population of cells within a defined location within a subject host, whereby the preparation is not delivered by administration into the circulatory system to be distributed throughout the body rather than specifically or mainly to the target tissue. It is expected that the administration may be by injection near the target tissue or into a vessel leading into the area to be treated.

The term “expressed” or “expression” as used herein refers to transcription from a gene to give an RNA nucleic acid molecule at least complementary in part to a region of one of the two nucleic acid strands of the gene. The term “expressed” or “expression” as used herein also refers to the translation from said RNA nucleic acid molecule to give a protein, a polypeptide or a portion thereof.

The term “flow cytometer” as used herein refers to any device that will irradiate a particle suspended in a fluid medium with light at a first wavelength, and is capable of detecting a light at the same or a different wavelength, wherein the detected light indicates the presence of a cell or an indicator thereon. The “flow cytometer” may be coupled to a cell sorter that is capable of isolating the particle or cell from other particles or cells not emitting the second light

The term “isolated” as used herein refers to a cell or population of cells removed from its/their natural environment such as a donor animal or tissue thereof, or removed from recognizably differing cells isolated from a host or tissue thereof.

The term “lentivirus” as used herein refers to a genus of retroviruses that can infect dividing and non-dividing cells. Several examples of lentiviruses include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates.

A lentiviral genome is generally organized into a 5′ long terminal repeat (LTR), the gag gene, the pol gene, the env gene, the accessory genes (nef, vif, vpr, vpu) and a 3′ LTR. The viral LTR is divided into three regions called U3, R and U5. The U3 region contains the enhancer and promoter elements. The U5 region contains the polyadenylation signals. The R (repeat) region separates the U3 and U5 regions and transcribed sequences of the R region appear at both the 5′ and 3′ ends of the viral RNA. See, for example, “RNA Viruses: A Practical Approach” Alan J. Cann, Ed., Oxford University Press, (2000); Narayan & Clements. Gen. (1989) Virology 70: 1617-1639; Fields et al., Fundamental Virology Raven Press. (1990); Miyoshi et al., (1998) J. Virol. 72: 8150-8157: U.S. Pat. No. 6,013,516.

The term “proliferative status” as used herein refers to whether a population of cells, and in particular stem cells or progenitor cells, or a subpopulation thereof, are dividing and thereby increasing in number, in the quiescent state, or whether the cells are not proliferating, dying or undergoing apoptosis. The term may also refer, for example, to a population of cells that constitute a body such as a tumor.

The terms “modulating the proliferative status” or “modulating the proliferation” as used herein refers to the ability of a compound to alter the proliferation rate of a population of stem cells, including muscle satellite cells, or progenitor cells. A compound may be toxic wherein the proliferation of the cells is slowed or halted, or the proliferation may be enhanced such as, for example, by the addition to the cells of a cytokine or growth factor, thereby increasing the proliferative rate.

The terms “fluorescent dye” and “fluorescent label” as used herein includes all known fluors, including rhodamine dyes (e.g., tetramethylrhodamine, dibenzorhodamine, see, e.g., U.S. Pat. No. 6,051,719); fluorescein dyes such as, but not limited to FITC; “BODIPY” dyes and equivalents.

The term “tissue” as used herein refers to a group or collection of similar cells and their intercellular matrix that act together in the performance of a particular function. The primary tissues are epithelial, connective (including blood), skeletal, muscular, glandular and nervous.

The term “primitive cell” as used herein refers to any stem, progenitor or precursor cell that may proliferate to form a population of differentiated colonic epithelial cells or tumor cells.

The term “stem cell” as used herein refers to pluripotent stem cells that, upon exposure to an appropriate cytokine or plurality of cytokines, may either differentiate into a progenitor cell of an epithelial cell lineage or proliferate as a stem cell population without further differentiation having been initiated.

The terms “progenitor” and “progenitor cell” as used herein refer to primitive cells that have differentiated to a developmental stage that, when the cells are further exposed to a cytokine or a group of cytokines, will differentiate further to an epithelial cell lineage. “Progenitors” and “progenitor cells” as used herein also include “precursor” cells that are derived from some types of progenitor cells and are the immediate precursor cells of some mature differentiated colonic epithelial cells. The terms “progenitor”, and “progenitor cell” as used herein include, but are not limited to, cells derived from the colon as normal tissue, or colonic tissue from a colonic cancer or colitic colon.

The terms “colon epithelial stem/progenitor cell” and “CS/PC” as used herein refer to a cell derived from the lining of the colon with the capacity for self-renewal that can differentiate into all (stem) or some (progenitor) of the cells that comprise the colon.

The terms “colon fibroblastic stromal cell(s)” and “CFStc(s)” as used herein refer to a fibroblast-like support cell isolated from the colon, from colitis, or from a colon cancer that provides a “niche” and support for the proper growth and development of CS/PC.

The term “substantially free” as used herein should be interpreted to be consistent with the empirical data presented in the examples. Also, a population is substantially free of cells dedicated to a particular lineage and/or cells carrying markers associated therewith. Preferably the population has less than 20%, more preferably less than 10%, e.g., less than 5%, of lineage committed cells.

The term “cytokine” as used herein refers to any cytokine or growth factor that can induce the differentiation of a stem cell to a progenitor or precursor cell and/or induce the proliferation thereof. Suitable cytokines for use in the present invention include, but are not limited to, stem cell factor, interleukin-1, interleukin-2, interleukin-3, interleukin-6, interleukin-7, interleukin-15, Flt3L, leukemia inhibitory factor, insulin-like growth factor, and insulin, and the like. The term “cytokine” as used herein further refers to any natural cytokine or growth factor as isolated from an animal or human tissue, and any fragment or derivative thereof that retains biological activity of the original parent cytokine. The cytokine or growth factor may further be a recombinant cytokine or a growth factor such as, for example, recombinant insulin.

The term “cytokine” as used herein further includes species-specific cytokines that while belonging to a structurally and functionally related group of cytokines, will have biological activity restricted to one animal species or group of taxonomically related species, or have reduced biological effect in other species.

The terms “patient” or “individual” are used interchangeably herein, and mean a mammalian subject to be treated, with human patients being preferred. In some cases, the methods of the disclosure find use in experimental animals, in veterinary application, and in the development of animal models for disease, including, but not limited to, rodents including mice, rats, and hamsters, as well as primates.

“Diagnostic” or “diagnosed” means identifying the presence or nature of a pathologic condition. Diagnostic methods differ in their sensitivity and specificity.

The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The term “prognosis” as used herein refers to the likelihood of a particular outcome in the development or treatment of a disease, and in particular the likelihood of the development of a colonic cancer.

The term “treatment” as used herein refers to the application or administration of a therapeutic agent described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The “treatment of cancer or tumor cells”, refers to one or more of the following effects: (1) inhibition, to some extent, of tumor growth, including, (i) slowing down (ii) inhibiting angiogenesis and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

The term “colitis” generally refers to inflammation of the colon, likely due to inflammatory bowel disease or a related condition.

The term “anaplasia” as used herein refers to a reversion of differentiation in cells and is characteristic of malignant neoplasms (tumors). Lack of differentiation is considered a hallmark of malignancy, implies loss of structural and functional differentiation of normal cells. Anaplastic cells display marked pleomorphism. The nuclei are characteristically extremely hyperchromatic (darkly stained) and large. Giant cells that are considerably larger than their neighbors may be formed and possess either one enormous nucleus or several nuclei (syncitia). Mitoses are often numerous and distinctly atypical. Anaplastic cells usually fail to develop recognizable patterns of orientation to one another (i.e. they lose normal polarity). They may grow in sheets, with total loss of communal structures, such as gland formation or stratified squamous architecture.

The term “adenocarcinoma” as used herein refers to a cancer originating in glandular tissue. This tissue is also part of a larger tissue category known as epithelial.

The term “sample” is used herein in its broadest sense. A sample including polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like. Examples of samples include feces, rectal mucus, blood, saliva, and biopsies.

The term “epithelial-mesenchymal transition” as used herein refers to a combination of cellular and molecular features in which epithelial cells lose several of their differentiated features and adapt more of an embryonic, undifferentiated phenotype. This change is characterized by a loss of polarity, decreased cell adhesion, and increased motility which render the epithelial cells more like mesenchymal cells. With these changes, the cells may express invasion, metastases and the acquisition of therapeutic resistance.

The present disclosure encompasses the isolation of CS/PCs and CFStcs from normal, colitis, and cancerous colon tissue, their propagation in in vitro and in vivo culture conditions, and their use in prognostic, diagnostic and clinical applications.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods involving isolating and culturing stem cells are described herein. Some techniques are generally known in the art and are described in detail, for example, in methodology treatises such as Culture of Human Stem Cells (Culture of Specialized Cells) by R. Ian Freshney et al., 1^{st} ed., Wiley-Liss Publishing, Hoboken, N.J., 2007; and Stem Cell Culture (Methods in Cell Biology), vol. 86, by Jennie P. Matter, 1^{st} ed., Academic Press, Burlington, Mass., 2008. Methods involving isolating and culturing CS/PC are described, for example, in Dalerba et al., (2007) Proc. Natl. Acad. Sci. USA 104: 10158-10163, and O'Brien et al., (2007) Nature 445:106-110. These references are herein incorporated by reference.

### CS/PC Spheroid Cultures (“Colonspheres”)

Described herein are methods of isolating CS/PCs from colitis and CRC and generating colonspheres therefrom. The “sphere” approach is a known approach for generating and propagating stem cells in culture conditions. In this method, single stem cells are grown in culture in a defined medium and are allowed to multiply in suspension, while retaining their ability to generate different cells types. The predominant shape of the cluster of cells is spheroid or rounded cluster, hence the ‘sphere” approach. The studies described herein are the first to generate colonspheres from ALDH1-positive cells from colitis samples and CRC biopsies, and the first to demonstrate the ability of these cells to generate different cell types of the colon crypt (a hallmark of cell “sternness”), the functional cellular unit of the colon.

### CS/PC Isolation and Characterization

Described herein are methods of isolating a population of mammalian pluripotent CS/PC from colitic samples and CRC biopsies. Adherent cell cultures of CS/PC and CFSt may be generated from primary colonic tissue and/or dissociated spheres by various enzymatic methods including trypsinization and/or the use of collagenase-dispase followed by gentle trituration to generate single cell suspensions. This is followed by seeding the cells in DMEM-containing medium supplemented with fetal bovine serum (FBS) at concentrations from 1 to 15%. The CFSt cells are easily separated from other cell types via their ability to rapidly adhere in culture conditions and removal of the medium followed by subsequent washes in isotonic salt solutions results in a highly enriched population of CFStcs. These cultures are then propagated and expanded to generate adherent CS/PC and CFSt cell lines.

In some embodiments, the CS/PC can be isolated based on their growing conditions. These methods are provided in U.S. Pat. No. 6,638,763 to Steindler et al., which is incorporated herein by reference, in its entirety. Two methods of xenografting have been used to establish the isolates. In the traditional methodology, tissues may be obtained from a patient undergoing a colectomy. This tissue can be implanted subcutaneously in the flank of NOD-SCID mice. When tumors grow, the resulting tissues are explanted and expanded into subsequent NOD-SCID hosts. Upon further growth, the tumors are dissociated and subjected to flow cytometry for enrichment. An alternative approach involves retrieval of the primary human colon tissue, followed by dissociation of the tumor first, and then subjecting the cell suspension to flow cytometry.

In the methods described herein, cell separation and cell adhesion can be manipulated using a variety of low adherent cell and tissue culture plasticware or other contact-limiting and contact-inhibiting factors. For example, single cell suspensions of bulk or sorted colon/colitis/cancer samples are grown in serum free defined medium supplemented with growth factors such as fibroblast growth factor 2, epidermal growth factor (EGF), insulin, transferring and sodium selenite. DMEM/F-12 with 15 mM HEPES, putrescine, progesterone, bovine serum albumin, and heparin has been used to generate the CS/PC cell lines described herein from colonic samples. Additional chemical-separating agents such as mercaptoethanol, physical separating agents such as methylcellulose, and anti-adhesives such as poly 2-hydroxyethyl methacrylate can be used to deter cell-cell and cell-substrate associates during the initial isolation of stem/precursor cells from the newly-dissociated colon is used to generate additional varieties of CS/PC. This allows the isolation of these cells from mature, differentiated tumors that are also dissociated during the tumor dissociation procedures. The mature, differentiated tumor cells cannot survive these anti-adhesion, anti-cell interaction procedures.

Thus, agents such as mercaptoethanol can be used in the first stage of isolation to help deter the survival of the more mature cellular elements (by deterring their clustering). At the same time, agents such as mercaptoethanol may have certain growth-promoting actions on the single stem/precursor cells that eventually proliferate to form these early sphere types, e.g., sarcospheres. Typically, a method for obtaining an isolated population of CS/PC can include culturing dissociated solid tumor cells on a non-adhesive substrate in suspension culture in defined media or media supplemented with fetal bovine serum and methyl cellulose, where culturing under conditions that inhibit cell-cell and cell-substrate interactions results in a substantially homogeneous population of pluripotent stem cells and multipotent colonic progenitor cells that are free from mature, differentiated cells.

CS/PCs as described herein are generally isolated by taking advantage of one or more stem cell markers (in particular, but not only, ALDH1) which, when bound to a detectable binding molecule, can be used to distinguish the bound cells from unbound cells, permitting separation and isolation. If the bound cells do not internalize the molecule, the molecule may be separated from the cell by methods known in the art. The molecule used for isolating the purified populations of CS/PC is advantageously conjugated with labels that expedite identification and separation. Examples of such labels include magnetic beads; biotin, which may be identified or separated by means of its affinity to avidin or streptavidin; fluorochromes, which may be identified or separated by means of a fluorescence-activated cell sorter (FACS, see below), and the like. Any technique may be used for isolation as long as the technique does not unduly harm the CS/PC.

The binding molecule can be attached to a solid support. Some suitable solid supports include nitrocellulose, agarose beads, polystyrene beads, hollow fiber membranes, magnetic beads, and plastic Petri dishes. For example, the binding molecule can be covalently linked to Pharmacia Sepharose 6 MB macro beads. Examples of the binding molecule include antibodies to: ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1. Other preferred markers include, but are not limited to: tenascins, proteoglycans, glycoproteins, glycolipids and other glycoconjugates that make up morphogenetic molecules and extracellular matrix molecules and their receptors, undulins and the like. For example, “glycosaminoglycan (GAG)-associated molecular interactions” is intended to include the binding of a GAG to, for example, a cell surface, secreted, or extracellular protein. This term also includes any subsequent results of such protein binding such as, for example, delayed proteolytic degradation or denaturing, changes in protein conformation (which may, for example, lead to alterations of biological activity), or catalysis of a reaction between two different proteins bound to the same or different GAGs on the same or different proteoglycans. Also included is the ability of certain GAGs, e.g., heparin sulfate, to modulate the interaction of a protein to another GAG, for example, FGF-2 (basic fibroblast growth factor) to its GAG cell receptor. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, von Willebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin; viral proteins (e.g., proteins from HIV, herpes complex) and other compounds which bind to GAG. The definition is intended to include the result of the binding of these factors to the GAG. For example, the binding of polypeptide growth factor to a GAG can result in cell proliferation, angiogenesis, inflammation, cancer, and other biologically important responses.

The exact conditions and duration of incubation for the solid phase-linked binding molecules with the crude cell mixture will depend upon several factors specific to the system used, as is well known in the art.

Cells that are bound to the binding molecule can be removed from the cell suspension by physically separating the solid support from the remaining cell suspension. For example, the unbound cells may be eluted or washed away with physiologic buffer after allowing sufficient time for the solid support to bind the CS/PC. The bound cells are separated from the solid phase by any appropriate method, depending mainly upon the nature of the solid phase and the binding molecule. For example, bound cells can be eluted by enzymatically “nicking” or digesting an enzyme-sensitive “spacer” sequence between the solid phase and an antibody (e.g., antibodies directed to: ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1). Suitable spacer sequences bound to agarose beads are commercially available from, for example, Pharmacia.

The eluted, enriched fraction of cells may then be washed with a buffer by centrifugation and preserved in a viable state at low temperatures for later use according to conventional technology. The cells may also be used immediately, for example by being infused intravenously into a recipient.

Methods for removing unwanted cells by negative selection are also known. For example, unwanted cells in a starting cell population are labeled by an antibody, or by a cocktail of antibodies, to a cell surface protein characteristic of Lin⁺ cells. The unwanted antibody-labeled cells are removed by methods known in the art. For example, the labeled cells can be immobilized on a column that binds to the antibodies and captures the cells.

Alternatively, the antibody that binds the cell surface proteins can be linked to magnetic colloids for capture of unwanted cells on a column surrounded by a magnetic field. This system is currently available through StemCell Technologies Inc., Vancouver, British Columbia, Canada. The remaining cells that flow through the column for collection are enriched in cells that do not express the cell surface proteins that the tetrameric antibodies were directed against. The antibody cocktail that can be used to deplete unwanted Lin⁺ cells can be custom made to include antibodies against lineage specific markers, such as, for example, CD2, CD3, CD4, CD5, CD8, CD10, CD11b, CD13, CD14, CD15, CD16, CD19, CD20, CD24, CD25, CD28, CD29, CD33, CD36, CD38, CD41, CD56, CD66b, CD66e, CD69, and glycophorin A. The desired cells that lack these markers are not lineage committed, i.e. they are Lin⁻.

A labeled binding molecule may be bound to the CS/PC, and the labeled cells separated by a mechanical cell sorter that detects the presence of the label, e.g., a fluorescence-activated cell sorter (FACS). FACS machines are commercially available. Generally, the following FACS protocol is suitable for this procedure: a Coulter Epics Eliter sorter is sterilized by running 70% ethanol through the systems.

The lines are flushed with sterile distilled water. Cells are incubated with a primary antibody diluted in Hank's balanced salt solution supplemented with 1% bovine serum albumin (HB) for 60 minutes on ice. The cells are washed with HB and incubated with a secondary antibody labeled with fluorescein isothiocyanate (FITC) for 30 minutes on ice. The secondary label binds to the primary antibody. Alternatively, the antibodies may be directly conjugated to a reporter. The sorting parameters, such as baseline fluorescence, are determined with an irrelevant primary antibody. The final cell concentration is usually set at one million cells per ml. While the cells are being labeled, a sort matrix is determined using fluorescent beads as a means of aligning the instrument. Once the appropriate parameters are determined, the cells are sorted and collected in sterile tubes containing medium supplemented with fetal bovine serum and antibiotics, usually penicillin, streptomycin and/or gentamicin and fungizone. After sorting, the cells are re-analyzed on the FACS to determine the purity of the sort.

Isolated cells from bulk tumors (e.g., colon cancer tumors) can be enriched for CS/PC populations. Preferably, cells are sorted with a FACS sorter using antibodies directed to any one of the stem cells markers described herein (e.g., ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1). The cells can be enriched using either positive or negative selection procedures.

### Identifying Candidate Therapeutic Compounds for CRC and Colitis

The presence of CS/PC isolated from colon cancer tumors indicates a role of these cells in the pathogenesis of such tumors. Without wishing to be bound by theory, the stem cells within a tumor have the ability, as do normal stem cells, to self renew. These few cells divide asymmetrically, producing an identical daughter cell and a more differentiated cell that goes on to comprise the vast majority of the tumor bulk. The stem-like cell is responsible for initiating and maintaining the growth of the tumor. In the case of colon cancer, chronic colitis has been associated with increased risk of malignant transformation. The studies described herein resulted in the discoveries that tumorigenic human colon cells can be isolated from colitis, that these tumorigenic human colon cells express ALDH1 at higher levels than do normal human colon cells, and that colitic epithelium, even in the absence of frank dysplasia or cancer, has the ability to foster tumorigenic growth.

The present disclosure encompasses methods of identifying candidate therapeutic compounds by culturing CS/PC expressing at least one marker selected from: ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1 with a candidate therapeutic agent; identifying candidate therapeutic agents that inhibit proliferation or growth, and/or lyse the CS/PC and/or inhibit CS/PC migration to a tumor and/or CS/PC differentiation in a tumor; wherein, growth and/or metastasis of a tumor is inhibited, and identifying a candidate therapeutic agent. A candidate therapeutic agent can include organic molecules, inorganic molecules, vaccines, antibodies, nucleic acid molecules, proteins, peptides and vectors expressing nucleic acid molecules.

One embodiment of the method of identifying candidate therapeutic compounds includes culturing CS/PC expressing at least one marker selected from: ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1 with the candidate compounds, and identifying those candidate compounds which bind to at least one of these markers. Other examples of markers include, but are not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-(3, IL-3); extracellular matrix components (e.g., laminins, fibronectins; thrombospondins, tenascins, collagens, von Willebrand's factor); proteases and anti-proteases (e.g., thrombin, TPA, UPA, clotting factors 1× and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin; viral proteins (e.g., proteins from HIV, herpes complex) and other compounds which bind to GAG.

A number of suitable assay methods to detect binding of test compounds to ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1 are known in the art, and include, but are not limited to, surface plasmon resonance (SPR)/BIACORE™, fluorogenic binding assays, fluid phase binding assays, affinity chromatography, size exclusion or gel filtration, ELISA, immunoprecipitation, competitive binding assays, gel shift assays, and mass spectrometry based methods, inter alia.

Another technique for identifying candidate therapeutic compounds (e.g., drug screening) provides for high throughput screening of compounds having suitable binding affinity to the protein of interest (see, e.g., Geysen et al. 1984, PCT application WO84/03564). In this method, large numbers of different small test compounds are synthesized on a solid substrate. The candidate compounds are reacted with ALDH1, ESA, Muc 1, Muc 2, CK19, c-kit, Oct 3/4, Nanog, Stro-1, or fragments thereof, and washed. Bound ALDH1, ESA, Muc 1, Muc 2, CK19, c-kit, Oct 3/4, Nanog, Stro-1 is then detected by methods well known in the art. Purified ALDH1, ESA, Muc 1, Muc 2, CK19, c-kit, Oct 3/4, Nanog, Stro-1 can also be coated directly onto plates for use in the aforementioned drug screening techniques. Alternatively, non-neutralizing antibodies can be used to capture the peptide and immobilize it on a solid support.

In some embodiments, methods described herein for identifying candidate therapeutic compounds include a first screen for compounds that bind to ALDH1. Compounds that are identified as binding to ALDH1 can then be used in a second screen to identify those compounds that inhibit a function of ALDH1. Alternatively, the first screen can be omitted and the compounds can simply be screened for their ability to inhibit a function of ALDH1 (e.g., to promote stem CS/PC cell preservation, proliferation, and survival). The ability of such compounds to treat tumors can be evaluated in a population of viable cells or in an animal, e.g., an animal model.

In one example of a method of identifying candidate therapeutic compounds for CRC and/or colitis, a candidate compound is administered to a model of the condition, e.g., contacting a cell (in vitro) model with the compound, or administering the compound to an animal model of the condition, e.g., an animal model of a condition associated with decreased CS/PC migration, such as cancer. The model is then evaluated for an effect of the candidate compound on the rate of migration in the model, and a candidate compound that decreases the rate of migration in the model can be considered a candidate therapeutic compound for the treatment of the condition. Such effects can include clinically relevant effects such as decreased tumor size or decreased tumor growth rate; decreased metastatic involvement or decreased rate of metastasis; decreased pain; increased life span; and so on. Such effects can be determined on a macroscopic or microscopic scale. Candidate therapeutic compounds identified by these methods can be further verified, e.g., by administration to human subjects in a clinical trial.

The candidate compounds utilized in the assays and methods described herein can be, inter alia, nucleic acids, small molecules, organic or inorganic compounds, antibodies or antigen-binding fragments thereof, polynucleotides, peptides, or polypeptides. For example, ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105 polypeptides or polynucleotides (e.g., ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1 polypeptide variants including truncation mutants, deletion mutants, and point mutants; nucleic acids including sense, antisense, aptamers, and small inhibitory RNAs (siRNAs) including short hairpin RNAs (shRNAs) and ribozymes) can be used as test compounds in the methods described herein. Alternatively, compounds or compositions that mimic the binding portions of ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1 can be used. A candidate compound that has been screened by an in vitro method described herein and determined to have a desired activity, e.g., binding of compounds to ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1 or affecting the functions of these molecules, for example, affecting growth, proliferation, migration of the CS/PCs to a tumor and the like, can be considered a candidate compound. A candidate compound that has been screened, e.g., in an in vitro or in vivo model, and determined to have a desirable effect on one or more inhibitory activities associated with treatment of cancer, (e.g., inhibition of proliferation, anti-angiogenic, apoptosis, decreased cell growth etc) can be considered a candidate therapeutic agent. Candidate therapeutic agents, once screened in a clinical setting, are therapeutic agents, and both types of agents can be optionally optimized (e.g., by derivatization), and formulated with pharmaceutically acceptable excipients or carriers to form pharmaceutical compositions.

Small molecule candidate compounds can initially be members of an organic or inorganic chemical library. The term “small molecule” as used herein refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht & Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, (1997) Curr. Opin. Chem. Bio. 1:60. In addition, a number of small molecule libraries are commercially available.

A candidate compound can have a structure that is based on an active fragment of ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1. For example, computer modeling methods known in the art can be used to rationally design a molecule that has a structure similar to an active fragment of ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1, or portions thereof.

The compounds can be optimized to improve their therapeutic index, i.e., increase therapeutic efficacy and/or decrease unwanted side effects. Thus, in some embodiments, the methods described herein include optimizing the test or candidate compound. In some embodiments, the methods include formulating a therapeutic composition including a test or candidate compound (e.g., an optimized compound) and a pharmaceutically acceptable carrier. In some embodiments, the compounds are optimized by derivatization using methods known in the art.

A candidate compound may include a polynucleotide that encodes ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1, or an active fragment thereof. In some embodiments, the compound is a polynucleotide that encodes an active fragment of ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1 that retain ligand binding activity.

RNAi is a remarkably efficient process whereby double-stranded RNA (dsRNA, also referred to herein as siRNAs, for small interfering RNAs, or ds siRNAs, for double-stranded small interfering RNAs) induces the sequence-specific degradation of homologous mRNA in animals and plant cells (Hutvagner and Zamore, (2002) Curr. Opin. Genet. Dev., 12: 225-232; Sharp, (2001) Genes Dev., 15: 485-490). In mammalian cells, RNAi can be triggered by duplexes of small interfering RNA (siRNA) (Chiu et al., (2002) Mol. Cell., 10: 549-561; Elbashir et al., (2001) Nature, 411: 494-498), or by micro-RNAs (miRNA), functional small-hairpin RNA (shRNA), or other dsRNAs which are expressed in vivo using DNA templates with RNA polymerase III promoters (Zeng et al., (2002) Mol. Cell, 9: 1327-1333; Paddison et al., (2002) Genes Dev., 16: 948-958; Lee et al., (2002) Nature Biotechnol., 20: 500-505; Paul et al., (2002) Nature Biotechnol., 20: 505-508; Tuschl, T., (2002) Nature Biotechnol., 20: 440-448; Yu et al., (2002) Proc. Natl. Acad. Sci. USA, 99: 6047-6052; McManus et al., (2002) RNA, 8: 842-850; Sui et al., (2002) Proc. Natl. Acad. Sci. USA, 99: 5515-5520).

The methods described herein can include the use of dsRNA molecules that are targeted to (i.e., bind to) ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1 mRNA (as well as any other suitable marker described herein). dsRNA can be delivered directly into cells in vivo or in vitro using methods known in the art, e.g., cationic liposome transfection, nanoparticles, and electroporation, or expressed in vivo or in vitro from recombinant DNA constructs that allow longer-term target gene suppression in cells, including mammalian Pol III promoter systems (e.g., H1 or U6/snRNA promoter systems (Tuschl (2002), supra) capable of expressing functional double-stranded siRNAs; (Bagella et al., (1998) J. Cell. Physiol. 177: 206-213; Lee et al., (2002), supra; Miyagishi et al., (2002), supra; Paul et al., (2002), supra; Yu et al., (2002), supra; Sui et al., (2002), supra).

### Methods of Screening Patients for CRC or an Increased Risk of CRC

Described herein are methods of screening patients for CRC and/or an increased risk of CRC. A typical method includes a first step of obtaining a sample (e.g., biopsy, blood, urine, feces, rectal mucus, etc.) from a patient who is being screened for CRC or an increased likelihood or risk of developing CRC, such as a patient having symptoms associated with colitis. Next, the sample is placed under appropriate conditions for dissociating cells. The dissociated cells are analyzed for ALDH1 by flow cytometry, frequency in blood, shed cells into stool, capacity to form spheres, and capacity to form xenografts. Other diagnostic methods include PCR, ELISAs, Western Blots, DNA sequencing, screening for epigenetic phenomena, and proteomics.

Expression or activity levels for ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1 also may be examined. Normal or standard values for ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1 expression are established by combining body fluids or cell extracts taken from normal mammalian subjects, preferably human, with antibody to, e.g., ALDH1, under conditions suitable for complex formation. The amount of standard complex formation may be quantitated by various methods, preferably by photometric means. Quantities of ALDH1 expressed in subject, control, and disease samples from biopsied tissues are compared with the standard values. Deviation between standard and subject values establishes the parameters for ALDH1 expression. Parameters studied include, but are not limited to, the below and those described throughout the specification.

### Treating a Colorectal Cancer Tumor in a Patient

Described herein are methods of treating a CRC tumor in a patient. In a typical embodiment, antibodies directed to ALDH1, ESA, Muc 1, Muc 2, CK19, c-kit, Oct 3/4, Nanog, activated Stat-3, CXCR4, CXCR1/2, CD133, SCA-1, Tra-1-60, CD44, CD73, CD90, CD105, and Stro-1 are covalently bound or fused to therapeutic effector domains and administered to a patient having a CRC tumor. Examples of therapeutic effector domains include, but not limited to: endostatin, angiogenin, angiostatin, chemokines, angioarrestin, angiostatin (plasminogen fragment), basement-membrane collagen-derived anti-angiogenic factors (tumstatin, canstatin, or arrestin), anti-angiogenic antithrombin III, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-b), vasculostatin, vasostatin (calreticulin fragment) and the like.

Cytolytic molecules that can be used to fuse to an antibody or fragment thereof, include, but are not limited to TNF-α, TNF-β, suitable effector genes such as those that encode a peptide toxin—such as ricin, abrin, diphtheria, gelonin, Pseudomonas exotoxin A, Crotalus durissus terrificus toxin, Crotalus adamenteus toxin, Naja naja toxin, and Naja mocambique toxin. (Hughes et al., (1996) Hum. Exp. Toxicol. 15: 443; Rosenblum et al., (1996) Cancer Immunol. Immunother. 42: 115; Rodriguez et al., (1998) Prostate 34: 259; Mauceri et al., (1996) Cancer Res. 56: 4311).

Also suitable are genes that induce or mediate apoptosis, such as the ICE-family of cysteine proteases, the Bcl-2 family of proteins, Bax, bclXs and caspases (Favrot et al., (1998) Gene Ther. 5: 728; McGill et al., (1997) Front. Biosci. 2: D353; McDonnell et al., (1995) Semin. Cancer Biol. 6: 53). Another potential anti-tumor agent is apoptin, a protein that induces apoptosis even where small drug chemotherapeutics fail (Pietersen et al., (2000) Adv. Exp. Med. Biol. 465: 153). Koga et al., ((2000) Hu. Gene Ther. 11: 1397) proposed a telomerase-specific gene therapy using the hTERT gene promoter linked to the apoptosis gene Caspase-8 (FLICE).

Also of interest are enzymes present in the lytic package that cytotoxic T lymphocytes or LAK cells deliver to their targets. Perforin, a pore-forming protein, and Fas ligand are major cytolytic molecules in these cells (Brandau et al., (2000) Clin. Cancer Res. 6: 3729; Cruz et al., (1999) Br. J, Cancer 81:881,). CTLs also express a family of at least 11 serine proteases termed granzymes, which have four primary substrate specificities (Kam et al., (2000) Biochim. Biophys. Acta 1477: 307). Low concentrations of streptolysin 0 and pneumolysin facilitate granzyme B-dependent apoptosis (Browne et al., (1999) Mol. Cell Biol. 19: 8604).

Other suitable effectors encode polypeptides having activity that is not itself toxic to a cell, but renders the cell sensitive to an otherwise nontoxic compound—either by metabolically altering the cell, or by changing a non-toxic prodrug into a lethal drug. Exemplary is thymidine kinase (tk), such as may be derived from a herpes simplex virus, and catalytically equivalent variants. The HSV tk converts the anti-herpetic agent ganciclovir (GCV) to a toxic product that interferes with DNA replication in proliferating cells.

The disclosure further provides for antibody fusion molecules comprising a modulatory or cytotoxic molecule fused to the F_{C} region, C_{H}1, C_{H}2 and/or C_{H}3, Fab, Fab′, F(ab′)₂, single chain Fv (scFv) and Fv fragments, as well as any portion of an antibody having specificity toward a desired target epitope or epitopes of a target protein such as, but not limited to, IL-6 or IL-8. Also preferred are antibodies or antibody fragments or to single chain, two-chain, and multi-chain proteins and glycoproteins belonging to the classes of polyclonal, monoclonal, chimeric, bispecific and hetero immunoglobulins (monoclonal antibodies being preferred); it also includes synthetic and genetically engineered variants of these immunoglobulins.

In another embodiment, carrier domains within the disclosure can be used to introduce an effector function to the molecule. For introducing an effector function to the molecule, the carrier domain can be a protein that has been shown to possess cytotoxic or immune response-stimulating properties. For instance, carrier domains for introducing a cytotoxic function to the molecule include a bacterial toxin, ricin, abrin, saporin, pokeweed viral protein, and constant region domains from an immunoglobulin molecule (e.g., for antibody dependent cell-mediated cytotoxicity). Molecules that contain a cytotoxic carrier domain can be used to selectively kill cells.

For introducing immune response-stimulating properties to a molecule, carrier domains within the disclosure include any molecule known to activate an immune system component. For example, antibodies and antibody fragments (e.g., CH₂—CH₃) can be used as a carrier domain to engage Fc receptors or to activate complement components. A number of other immune system-activating molecules are known that might also be used as a carrier domain, e.g., microbial superantigens, adjuvant components, lipopolysaccharide (LPS), and lectins with mitogenic activity. Other carrier domains that can be used to introduce an effector function to the molecule can be identified using known methods. For instance, a molecule can be screened for suitability as a carrier domain by fusing the molecule to an anti-angiogenic agent and testing the molecule in in vitro or in vivo cell cytotoxicity and humoral response assays.

### Kit for Screening Patients for Colorectal Cancer or an Increased Risk of Colorectal Cancer

Described herein are kits for screening patients for CRC or an increased risk of CRC. A typical kit includes a reagent for detecting ALDH1 in cells, their frequency and/or distribution, and instructions for use. Any reagent suitable for detecting ALDH1 in cells can be used. In one example of a kit, the reagent for detecting ALDH1 is an antibody that specifically binds ALDH1 within the epithelium from the colorectum or in the blood. In another example of a kit, a FLOW cytometry-based screening kit is used to isolate cells from stool and then stain the cells with ALDH1-reactive fluorescent agent. In a further embodiment, a kit for use by a physician (e.g., a pathologist) includes a means for isolating cells and a means for performing histological staining of cells for ALDH1.

One aspect of the present disclosure encompasses an isolated mammalian pluripotent colon epithelial stem/progenitor cell (CS/PC), or a population of said cells, wherein each CS/PC may comprise a detectable marker, wherein the detectable marker is aldehyde dehydrogenase 1 (ALDH1), and wherein the isolated population of mammalian pluripotent CS/PCs is substantially free of cells that do not have the detectable ALDH1 marker.

In embodiments of this aspect of the disclosure, the isolated mammalian pluripotent colon epithelial stem/progenitor cell (CS/PC), or population of said cells may be further characterized by further comprising at least one detectable marker selected from the group consisting of: ESA, c-kit, Oct 3/4, Nanog, activated Stat-3, CXCR4, CXCR1/2, CD133, SCA-1, Tra-1-60, CD44, CD73, CD90, CD105, and Stro-1.

In various embodiments of this aspect of the disclosure the isolated mammalian pluripotent colon epithelial stem/progenitor cell (CS/PC), or population of said cells, may further comprise at least one of the detectable markers CD133 and CD44.

In embodiments of this aspect of the disclosure, the isolated mammalian pluripotent colon epithelial stem/progenitor cell (CS/PC), or population of said cells, can be isolated from a normal colon, a cancerous colon, or a colitic colon.

In embodiments of the disclosure, the isolated mammalian pluripotent colon epithelial stem/progenitor cell (CS/PC), or population of said cells, may be isolated from a cancerous colon.

In other embodiments of the disclosure, the isolated mammalian pluripotent colon epithelial stem/progenitor cell (CS/PC), or population of said cells, may be isolated from a colitic colon.

In one embodiment of this aspect, the isolated mammalian pluripotent colon epithelial stem/progenitor cell (CS/PC), or population of said cells, when isolated from the colitic colon, and when engrafted into a recipient animal, can form an anaplastic mass. In one embodiment, when said cells are serially passaged from the anaplastic mass into a recipient animal or series of animals, they can form an adenocarcinoma, wherein the cells of the adenocarcinoma have the characteristics of a colon cancer cell.

Another aspect of the present disclosure provides embodiments of a population of cultures mammalian pluripotent colon epithelial stem/progenitor cells (CS/PCs) according to any of the above embodiments, wherein the population of CS/PCs may be cultured under in vitro conditions, thereby forming a cell-based spheroid.

Yet another aspect of the present disclosure provides for a fibroblast-epithelial cell co-culture comprising an isolated mammalian pluripotent colon epithelial stem/progenitor cell (CS/PC), or population of said cells, and an isolated mammalian colonic stromal fibroblast cell (CS/PC), or population of said cells.

In embodiments of this aspect of the disclosure the each CS/PCs of the fibroblast-epithelial cell co-culture may comprise a detectable marker, wherein the detectable marker is aldehyde dehydrogenase 1 (ALDH1), and wherein the isolated population of mammalian pluripotent CS/PCs can be substantially free of cells that do not have the detectable ALDH1 marker.

In the various embodiments of this aspect of the disclosure, each CS/PC of the fibroblast-epithelial cell co-culture may be further characterized by further comprising at least one detectable marker selected from the group consisting of: ESA, c-kit, Oct 3/4, Nanog, activated Stat-3, CXCR4, CXCR1/2, CD133, SCA-1, Tra-1-60, CD44, CD73, CD90, CD105, and Stro-1.

In one embodiment of the disclosure, the co-culture may be cultured in in vitro tissue culture, wherein the growth conditions generate cell spheroids.

In another embodiment, the co-culture may be cultured in a recipient subject animal.

Still another aspect of the present disclosure encompasses methods of isolating a mammalian pluripotent colon epithelial stem/progenitor cell (CS/PC), or a population of said cells, from an animal or human colon, wherein each CS/PC comprises a detectable marker, wherein the detectable marker is aldehyde dehydrogenase 1 (ALDH1), said method comprising the steps of: (i) obtaining a tissue sample from the colon of an animal or human subject; (ii) disrupting the tissue sample, thereby obtaining a cell suspension, wherein the cell suspension is substantially comprised of single cells; (iii) contacting the cell suspension with at least one ligand species capable of selectively binding to a cell marker, wherein each cell marker, or combination of markers selectively identifies a mammalian pluripotent colon epithelial stem/progenitor cell (CS/PC), and wherein each ligand species comprises a detectable label; (iv) identifying a cell or population of cells binding to the ligand species or combination of ligand species; and (v) isolating the identified cell or population of cells selectively binding to the ligand species or combination of ligand species from a population of cells not selectively binding to the ligand species or combination of ligand species, thereby obtaining an isolated population of mammalian pluripotent colon epithelial stem/progenitor (CS/PC) cells.

In embodiments of this aspect of the disclosure, the method may further comprise the step of: culturing the isolated population of mammalian pluripotent colon epithelial stem/progenitor (CS/PC) cells in a medium under proliferative conditions.

In other embodiments, the method may further comprise the step of: culturing the cell suspension in a medium under conditions wherein the cells are allowed to proliferate to form a spheroid population of cells.

In other embodiments, the method may further comprise the step of: culturing the cell suspension in a medium under conditions wherein the cells are allowed to proliferate to form an adherent layer of cells.

In still other embodiments, the method may further comprise the step of: culturing the cell suspension in a medium under conditions wherein the cells are co-cultured with a population of isolated colon stromal fibroblasts. In these embodiments, the at least one ligand species may be selected from the group consisting of ALDH1, ESA, c-kit, Oct 3/4, Nanog, activated Stat-3, CXCR4, CXCR1/2, CD133, SCA-1, Tra-1-60, CD44, CD73, CD90, CD105, and Stro-1.

In the embodiments, at least one ligand species may selectively bind to aldehyde dehydrogenase 1 (ALDH1).

In some embodiments, the isolated cells binding to a ligand species selectively binding to a marker of a CS/PC but not to a non-CS/PC may be isolated by a cell sorter.

In the embodiments of this aspect of the disclosure, the medium may comprise at least one cell separation compound selected from the group consisting of: a chemical-separating compound, a physical-separating compound, and an anti-adhesive compound.

In the embodiments of this aspect, the medium can be serum-free, and comprise at least one growth factor.

In some embodiments of this method of the disclosure, the tissue sample may be from a cancerous colon.

In other embodiments of this method of the disclosure, the tissue sample may be from a colitic colon, where the CS/PCs, when isolated from the colitic colon, when engrafted into a recipient animal, can form an anaplastic body, and, when said cells are serially passaged from the anaplastic body into a recipient animal or series of animals, can form an adenocarcinoma, where the cells of the adenocarcinoma have the characteristics of a colon cancer cell.

Yet another aspect of the present disclosure encompasses methods for producing self-renewing pluripotent CS/PC clones comprising the steps of: culturing colonspheres; dissociating colonspheres into single cells; culturing said single cells to near confluence and harvesting; reseeding harvested cells into suspension (non-adherent) cultures; identifying self-renewing cells by formation of secondary spheres; and isolating self-renewing pluripotent CS/PC clones.

In this aspect of the disclosure, the self-renewing pluripotent CS/PC clones may express at least one of: ALDH1, ESA, c-kit, Muc1, Muc2, CK19, Oct 3/4, Nanog, activated Stat-3, CXCR4, CXCR1/2, CD133, SCA-1, Tra-1-60, CD44, CD73, CD90, CD105, vimentin, and Stro-1.

Another aspect of the disclosure encompasses methods for determining the prognosis for a patient for developing a colon cancer, the method comprising: (i) obtaining an isolated tissue sample from the colon of an animal or human patient; (ii) obtaining a tissue section from the isolated tissue section; and (iii) detecting the presence of at least one marker in the tissue section, where the marker or plurality of markers indicate the presence of a pluripotent colon epithelial stem/progenitor cell (CS/PC), thereby indicating the prognosis of the patient for developing a colon cancer.

In embodiments of this aspect of the disclosure, the marker or plurality of markers can be selected from the group consisting of: ALDH1, ESA, c-kit, Mud, Muc2, CK19, Oct 3/4, Nanog, activated Stat-3, CXCR4, CXCR1/2, CD133, SCA-1, Tra-1-60, CD44, CD73, CD90, CD105, vimentin, and Stro-1.

In some embodiments, the plurality of markers may comprise ALDH1 and at least one marker selected from the group consisting of: ESA, c-kit, Muc1, Muc2, CK19, Oct 3/4, Nanog, activated Stat-3, CXCR4, CXCR1/2, CD133, SCA-1, Tra-1-60, CD44, CD73, CD90, CD105, vimentin, and Stro-1.

In certain embodiments, the plurality of markers comprises ALDH1 and at least one marker selected from the group consisting of: ESA, CD133, CD44, and vimentin.

In one embodiment, the plurality of markers comprises ALDH1 and ESA.

In another embodiment, the plurality of markers comprises ALDH1 and vimentin.

In embodiments of this aspect of the disclosure, the methods may further comprise the steps: disrupting the tissue sample, thereby obtaining a cell suspension, where the cell suspension is substantially comprised of single cells; contacting the disrupted tissue sample with at least one labeled ligand species capable of selectively binding to a cell marker indicative of a mammalian pluripotent colon epithelial stem/progenitor cell (CS/PC); isolating a pluripotent colon epithelial stem/progenitor (CS/PC) cell or a population of said cells from the disrupted tissue sample by isolating a labeled ligand species bound to a cell or cells of the disrupted tissue sample, thereby obtaining an isolated population of mammalian pluripotent colon epithelial stem/progenitor (CS/PC) cells; culturing the isolated population of pluripotent colon epithelial stem/progenitor (CS/PC) cells in a medium under conditions favorable for the proliferation of pluripotent colon epithelial stem/progenitor (CS/PC) cells; and determining whether the cultured cells comprise colon epithelial stem/progenitor (CS/PC) cells; thereby determining the prognosis for the patient developing a colonic cancer.

In the embodiments of this aspect, the tissue sample can be isolated from a patient having the symptoms of a colitis, where the CS/PCs, when isolated from the colitic colon, when engrafted into a recipient animal, can form an anaplastic body, and when said cells are serially passaged from the anaplastic body into a recipient animal or series of animals, can form an adenocarcinoma, where the cells of the adenocarcinoma have the characteristics of a colon cancer cell.

In other embodiments, the method may further comprise the steps of: culturing the cell suspension in a medium under conditions where the cells are allowed to proliferate to form a spheroid population of cells; and identifying the cells of the spheroid as colon epithelial stem/progenitor (CS/PC) cells; thereby determining prognosis for the patient developing a colonic cancer.

In yet other embodiments, the method may yet further comprise the steps of: administering the isolated cells to a recipient animal and allowing the animal to develop a tumor; and identifying the cells of the developed tumor as colon epithelial stem/progenitor (CS/PC) cells; thereby determining prognosis for the patient developing a colonic cancer.

Another embodiment of the method of this aspect of the disclosure may further comprise the step of: co-culturing the cell suspension with colonic stromal fibroblasts isolated from the patient.

This embodiment may yet further comprise determining the presence of an epithelial-mesenchymal transition event in the spheroid, wherein the presence of the epithelial-mesenchymal transition event indicates that at least one of the spheroid cells is metastatic.

This embodiment may also yet further comprise determining the presence of an epithelial-mesenchymal transition event in the tumor, wherein the presence of the epithelial-mesenchymal transition event indicates that at least one of the spheroid cells is metastatic.

Still yet another aspect of the present disclosure encompasses kits comprising a container comprising a detectable ligand, or a combination of detectable ligands, where each species of detectable ligand specifically binds to an individual marker, wherein a marker alone, or in combination with at least one other marker, identifies a colonic stem/progenitor cell from a population of cells of a mammal, and instructions for the use of the detectable ligand or combination of markers to identify a colonic stem/progenitor cell from a population of cells of a mammal.

Embodiments of this aspect of the disclosure may further comprise instructions for the use of the detectable ligand, or combination of detectable ligands, for the isolation of a colonic stem/progenitor cell from a population of cells of a mammal.

In embodiments of the kits of this aspect of the disclosure, the at least one detectable ligand may be selected from the group consisting of: ALDH1, ESA, c-kit, Muc1, Muc2, CK19, Oct 3/4, Nanog, activated Stat-3, CXCR4, CXCR1/2, CD133, SCA-1, Tra-1-60, CD44, CD73, CD90, CD105, vimentin, and Stro-1

Another aspect of the disclosure is a method of identifying a candidate therapeutic compound effective in reducing the proliferative status of a colonic stem/progenitor cell of a mammal, comprising the steps of: culturing a population, or plurality of populations, of isolated CS/PCs, with a plurality of candidate therapeutic agents, wherein each population of CS/PCs expresses at least one marker selected from the group consisting of: ALDH1, ESA Oct 3/4, Nanog, activated Stat-3, CXCR4, CXCR1/2, CD133, SCA-1, Tra-1-60, CD44, CD73, CD90, CD105, vimentin, and Stro-1; and identifying a candidate therapeutic agent that reduces the proliferation status of a culture of a population of CS/PCs.

Another aspect of the present disclosure provides methods of modulating the proliferative status of a colonic cancer cell comprising: contacting a colonic cell with an effective amount of an agent, where the agent selectively binds to a cell surface marker, and where the agent when bound to the cell surface marker inhibits binding of a factor to the cell, thereby modulating the proliferative status of the colonic cell.

In one embodiment of this aspect of the disclosure, the colonic cell is a colon epithelial stem/progenitor (CS/PC) cell comprising the marker ALDH1.

In some embodiments, the cell surface marker is a receptor molecule.

In certain embodiments, the receptor molecule selectively binds to IL-6 or IL-8.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

### Immunohistochemistry

Normal colon tissues were retrieved from one of two sources: either control tissue at least 10 cm away from a malignancy, or from patients presenting for resection of benign disorders such as slow-transit constipation. Routine assays for immunohistochemistry and immunofluorescence were (marker-specific antibody dilutions and designations in parentheses): ALDH1 (1:100 BD Biosciences), CD44 (1:50 Clone G44-26, Bectin-Dickinson), IL-8R (1:100 Abcam) and MUC2 (1:100 clone CCP58, Zymed) were completed from paraffin embedded sections. Either MOM (Vector labs for ALDH1) or ARK (DAKOCytomation, for MUC2 or CD44) antigen retrieval methods were used in xenograft sections. Other antibodies included CD30 (DAKO), CDX2 (1:50 Abcam), CD45 (DAKO), CAM 5.2 (BD Biosciences), AE1/AE3 (Millipore), CK19 (1:100 Abcam), CK7 (DAKO) CD2 (DAKO), CD3 (DAKO), and S100 (DAKO). The stromal isolates were stained with SMA (1:600 R&D Systems). For such immunohistochemistry and immunofluorescence assays on sphere, the spheres were concentrated into a 1% low melting point agarose plug prior to embedding in paraffin.

### Example 2

### Xenograft Assays

For colon cancer tissue, cancer and matched normal tissues were retrieved from the operating room and maintained under sterile conditions. For colitic tissues, both distal and proximal regions of the colon were implanted. All tissue retrieval was completed under pathologic supervision.

Tissues were minced and placed subcutaneously into the flanks of NOD-SCID mice. For cellular xenografts, the selected numbers of cells were pelleted post flow cytometry. The pellets were resuspended in Matrigel (BD Biosciences) such that the total volume for injection was not greater than 100 μl. The opposite flank of an animal received the control injection. For the antibody blocking experiments, commercially available pellets (Innovation Research of America, Sarasota, Fla.) were embedded with either anti-IL-8 or anti-IL-6 (R&D Systems, 5 g/day) or placebo (control) and implanted one day prior to placement of the xenografts. To account for variability, at least 8 mice per condition were initiated. Mice were monitored daily for tumor growth and measured at least weekly to establish tumor volume.

### Example 3

### Flow Cytometry Sorting

The tissues were digested with collagenase. Antibodies were added at a concentration of 1:100 and maintained for 30 minutes on ice. The cell pellets were washed with HBSS/2% FBS, and in circumstances where a secondary antibody is required, the cells are resuspended in the appropriate secondary antibody.

For the identification of CD44 and ESA antigens, the anti-CD44 allophycocyanin (APC, Pharmingen, Franklin Lakes, N.J.) and anti-epithelial specific antigen (ESA)-FITC (Biomeda, Foster City, Calif.) were employed at a dilution of 1:40. Nonviable cells were eliminated by using the viability dye, DAPI, just prior to submission for flow cytometry. Murine cells were eliminated by staining with H2K^{d} (Southern biotech). Flow cytometry was performed on the FACS Aria (BD Immunocytometry Systems, Franklin Lakes, N.J.). Side scatter and forward scatter profiles was utilized to eliminate cell doublets. Cells were routinely subjected to double sorting, and reanalyzed for purity, which was typically greater than 97%. The ALDEFLUOR™ kit (Stem Cell Technologies, Vancouver, Canada) was used to identify the ALDH1 positive population. Controls included the viability stain, DAPI, the ALDEFLUOR™-stained cells treated with inhibitor of the enzyme (DEAB), and staining with the secondary antibody alone.

### Example 4

### Sphere Culture

Flow cytometry was utilized to sort cells from xenografts either from colorectal oncogenic or colitic sources. Isolated cells were placed into a low attachment six well plate at a density of 2000-10,000 cells/well. These cells were suspended in a serum-free medium containing DMEM/F12 (Gibco), 10 nM progesterone (Sigma), glutamine, Gibco), 10 μg/ml insulin (Sigma), 13 μg/ml transferrin (Sigma), 15 nM sodium selenite (Sigma), 4 mg/ml BSA (Sigma), 50 μM putrescine (Sigma), and 15 mM HEPES (Gibco), supplemented with 10 ng/ml FGF and 20 ng/mIEGF (Sigma).

### Example 5

### Isolation and Maintenance of Primary Stromal Isolates

Tissues from colitic, colon cancer, and matched tissues were minced and digested with collagenase type 1A (Sigma) for 30 minutes at 37° C. The resulting suspension was plated in serum containing media (DMEM 10% FBS, Gibco). For these experiments, successful isolates were employed in passages 4-9. Prior to use, observation of phenotype and staining with a smooth muscle actin was used to confirm these cells as fibroblasts. For these experiments, successful isolates were employed in passages 4-9. For comparison, normal colon fibroblasts (CRL 1541) and colon cancer fibroblasts (CRL7213) were purchased from the American type culture collection (ATCC, Manassas, Va.)

### Example 6

### Cytokine Array Analysis

Conditioned media was harvested from stromal isolates. Conditioned media was harvested after plating of successful isolates at a density of 10⁵ cellsin a well of a 24-well tissue culture treated plate. After the cells had adhered, the media was removed and replaced with fresh media in the absence of serum. After 24 hours 37° C., 5% CO₂, the overlying media was harvested for use in the cytokine/chemokine array. The Chemicon cytokine array (Human Cytokine Array III, Millipore) was conducted according to the manufacturer's instructions.

### Example 7

### Statistical Analysis

The incidence of ALDH1 expressing epithelium was enumerated in normal versus colitic epithelium. In total, at least 5000 epithelia per disease type were counted. The data were analyzed as a two sample t-test with p<0.05 taken as significant. For comparison of tumor latencies, growth rates, and size, ANOVA using Duncan's multiple comparison procedure at the 0.05 significance level was determined.

### Example 8

### Markers for Colon Cancer-Initiating Cells are Present in Normal Colon and have Expanded Expression in Colon Cancer

Immunohistochemistry employing the S1700 antigen retrieval buffer (DAKO), and antibodies for CD133 (Miltenyi Biotec, 1:5), CD44 (BD Biosciences, 1:200), and ALDH1 (BD Biosciences, 1:100) was carried out with DAB as the chromogen.

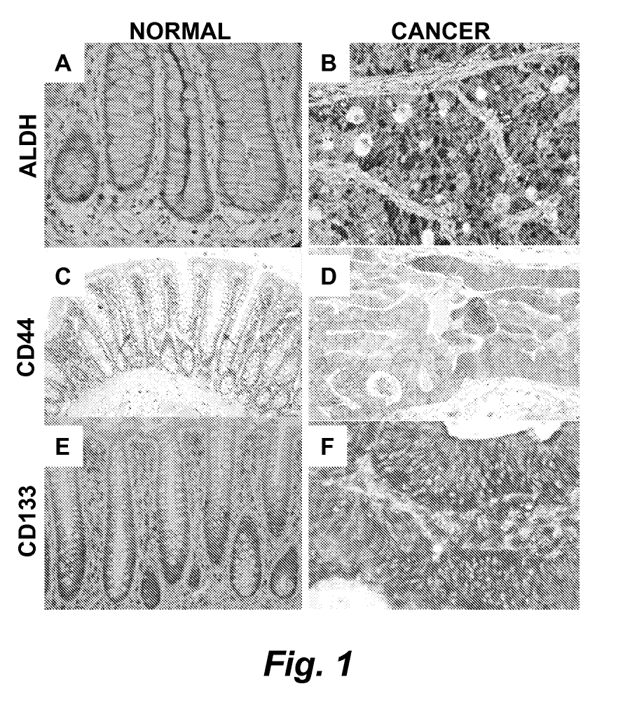

Expression of ALDH1 was limited to rare cells at the base of the crypt in normal colonic epithelium, as shown in FIG. 1A (N=30). The expression of ALDH1 in colorectal cancer was often expanded to the entire lesion, especially at the invasive front (FIG. 1B, N=30).

In normal colonic epithelium, the expression of CD44 often constituted the basal half to third of the crypt (FIG. 1C, arrows). Invasive colon cancer reveals CD44 expression at the basal and luminal surfaces (FIG. 1D).

Normal colonic epithelium expresses CD133 at basal third (arrows) (FIG. 1E). The expression of CD133 was more challenging to detect, but when detectable, strongly mimicked the pattern seen for CD44, with the bottom half of the crypt bearing expression in the normal colonic epithelium (FIG. 1E, arrows). In colorectal cancer, the expression of CD133 was expanded throughout the tumor (invasive colon cancer expresses CD133 throughout the cytoplasm) (FIG. 1F).

The expression of ALDH1 in the normal colon crypt, therefore, is limited to rare cells at or near the base of the crypt, possibly denoting the normal colon CS/PC. The immunohistological expression of CD44, CD133 and ALDH1 is expanded in colorectal cancer.

### Example 9

### ALDH1 Positive Cells are Present Near the Base of the Normal Colonic Crypt

The expression of ALDH1 overlaps ESA (epithelial surface antigen) at the invasive front of colon cancer. The expression of ESA in colonic epithelium can cover the entire epithelium and thus, would be an unlikely marker allowing increased enrichment for colon cancer or normal colon CS/PCs. While not wishing to be bound by theory, the expression of ALDH1 in a small minority of cells at the base of the normal colonic epithelial crypt is more consistent with the genesis and regeneration of colonic epithelia. To test whether the expression of ALDH1 occurs only in a subset of the colonic epithelium, immunofluorescence was performed.

Dual immunofluorescence for ALDH 1 and ESA was conducted using standard conditions. Antigen retrieval with the S1700 buffer (DAKO), followed by ESA-HTC staining. This stain was followed with ALDH1 and a secondary phycoerythrin antibody (Goat anti-mouse, 1:100), and then cover-slipping with DAPI prior to examination by fluorescence microscopy.

Normal colon immunofluorescence revealed nuclear staining, membranous ESA staining throughout the crypt and only rare ALDH1 cytoplasmic staining.

Invasive colon cancer, however, demonstrated nuclear staining, increased expression of ESA, and expanded expression of ALDH1.

Expression of ALDH1 was limited to rare cells at the base of the crypt, surrounded by an ESA membranous stain in normal colon. This expression was expanded in colonic malignancy, especially at the leading edge of the tumor. The expression of ALDH1 in colonic epithelium, therefore, consists of rare cells, at the base of the crypt, which are a subset of ESA-positive cells. Further, this expression is expands to encompass the invasive edge of colorectal cancer.

### Example 10

### ALDH1 Expression is Limited to Rare Cells at the Base of the Normal Crypt which Expands and Overlaps CD44 in Malignancy

CD44 has been reported to increase the enrichment of putative colon cancer stem cells. However, the expression of CD44 typically is concentrated to the basal third—lower half of the crypt. Co-expression with ALDH1 was examined.

Dual immunofluorescence was used as in Example 9, above. The expression of CD44 was determined using anti-CD44 (BD Biosciences), followed by FITC-conjugated secondary antibody. The expression of ALDH1 was determined using the anti-ALDH1 antibody followed by the PE-conjugated secondary antibody.

As in routine immunohistochemistry, CD44 expression was present in the bottom third to lower half of the crypt, and expanded in colorectal cancer. The expression of ALDH 1 remained restricted to a small minority of cells at the base of the crypt in normal colonic epithelium, a subset of CD44, and expanded at the invasive edge of colon cancer.

Thus, ALDH1 expression is a more exclusive marker for putative normal colon CS/PCs and colon cancer initiating cells (e.g., CS/PC) than is CD44 alone.

### Example 11

### Colon Cancer Cells Isolated Using ESA and ALDH1 Directly from Primary Human Colon Cancer have the Capacity to Recapitulate the Primary Adenocarcinoma

Xenografts from colon cancer cell lines often require 1-5×10⁶ cells to generate a tumor. It was examined whether flow sorting of cells directly from a primary colorectal cancer by selecting for ALDH1 intracellular enzyme marker expression would facilitate tumorigenesis in the absence of an initial passage of whole primary tissue fragments. Previous assays have used membranous (surface) markers, the function of one of which, CD133, is currently unknown. In contrast to ALDH1, since both CD133 and CD44 are on the cell surface, there is always the possibility that these markers can be damaged, lost, or decreased in the tissue dissociation process, such as by trypsinization.

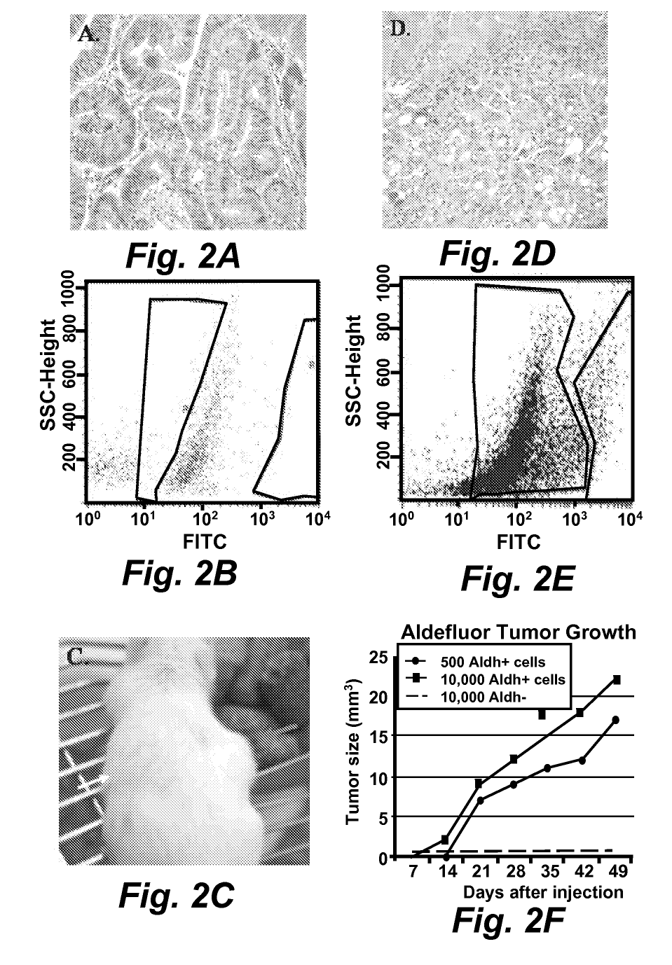

Primary colorectal cancerous tissue was harvested fresh from the operating room and was digested with collagenase. The tissue digest was stained for both ALDH1, using the ALDEFLUOR™ kit (Stemcell Technologies, Vancouver, British Columbia) and ESA. ESA was used to eliminate potential ALDH1 overlap with stromal cells. Serial dilutions of ALDH1-positive cells were injected into the flanks of NOD-SCID mice. The results of this experiment are shown in FIGS. 2A-2F.

The typical histology of a primary colon cancer is shown in FIG. 2A. Flow cytometry of cells digested from the primary tumor revealed a population of cells that were ESA^{high}/ALDH1^{high}, as shown in FIG. 2B. By 4 weeks after injection, a mass was clearly visible in the right flank of the recipient NOD-SCID mouse host, where 1000 of the ESA^{high}/ALDH1^{high} cells had been injected, as shown in FIG. 2C. The opposing flank of the recipient animal, which received 1000 ESA^{high}/ALDH1^{high}% cells had no growth at three weeks, nor for the remainder of the period of observation.

Histologically, the adenocarcinoma was easily discernible (see FIG. 2D). In subsequent sorting of the cells of the xenograft, both ALDH1 positive and negative populations were observed by flow cytometry, as shown in FIG. 2E and, therefore, resembling the original cellular populations. The latency of tumor growth depended on the cell numbers injected, as shown graphically in FIG. 2F.

Accordingly, ALDH1 may be used as a determinant marker to facilitate the isolation of colon cancer initiating cells from primary colon cancers. The xenografts resulting from such sorted cells resemble the primary (parent) tumors regarding their flow characteristics and histology. Tumor latency was directly related to the number of cells injected. It was further found that few as 25 ALDH1^{high}/ESA^{high} cells were capable of engraftment and tumorigenicity.

### Example 12

### Expression of CD44 and ALDH1 is Increased in Colitis and in Colon Cancer

Whether or not ALDH1 expression differed between normal colonic epithelium, colitic tissue, and colon cancer was examined. Immunohistochemistry detection of ALDH1 (AEC chromogen, 1.100, BD Biosciences) was used.

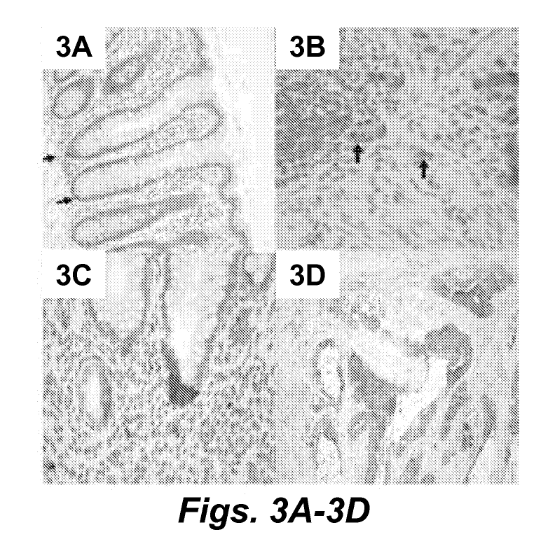

ALDH1 expression was seen in single cells at the base of the crypt of a normal colon (FIG. 3A), while ALDH1 expression is expanded in colitis and sporadic cancer tissue, as shown in FIGS. 3B and 3C. Significantly, this expression is dramatically increased in the case of colitis-associated cancer, as shown in FIG. 3D. The expression of ALDH1 in the normal tissues is limited to rare cells at the base of the crypt and is more specific than CD133 or CD44 (as shown in the histological results shown in FIGS. 1A-1D, and discussed in Example 8, above). The expression is enhanced throughout the epithelium in the case of colitis-associated cancer, indicating that the marker ALDH1 may serve as a more significant marker for colonic and colitic sternness.

### Example 13

### Tumorigenic Human Colon Cells have been Isolated from Colitic Colon Tissue

Chronic colitis has been associated with an increased risk of malignant transformation. Techniques such as those used to isolate putative colon cancer stem cells (see for example, O'Brien et al., (2007) Nature 445: 106; Ricci-Vitiani et al., (2007) Nature 445: 111; Dalerba et al. (2007) Proc. Natl. Acad. Sci. USA 104: 10158) have been used. Routine examination of a colitic specimen includes: 1: histological examination of areas of gross abnormalities, 2: in those cases where dysplasia has been diagnosed preoperatively, increased sampling for evidence of dysplasia or an occult invasive malignancy, and 3: sampling of remaining, apparently non-colitic, areas of the colon.

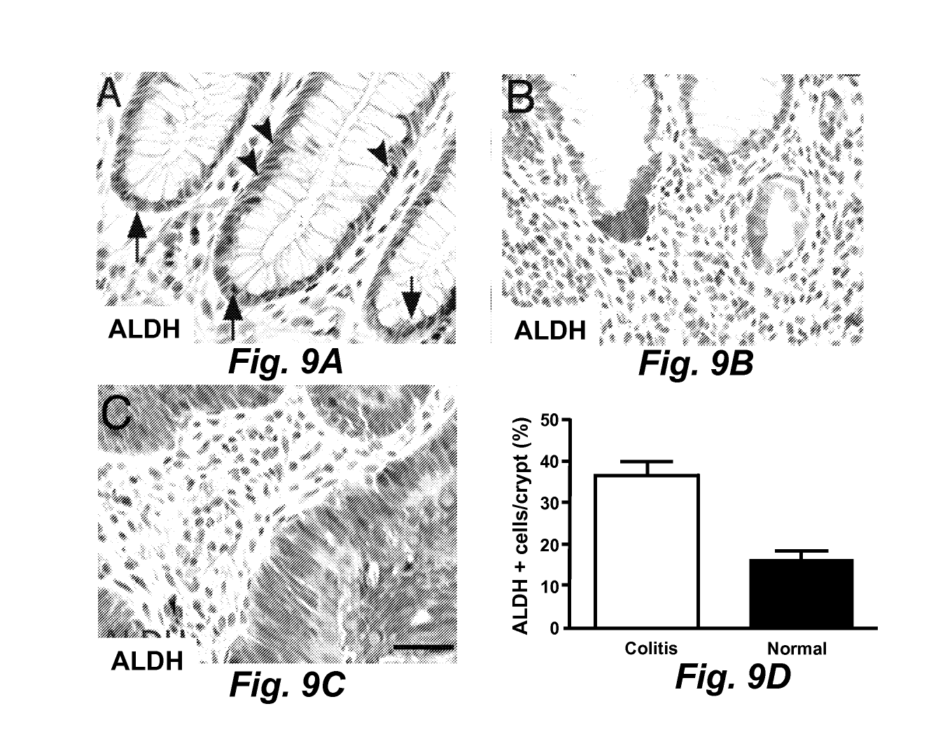

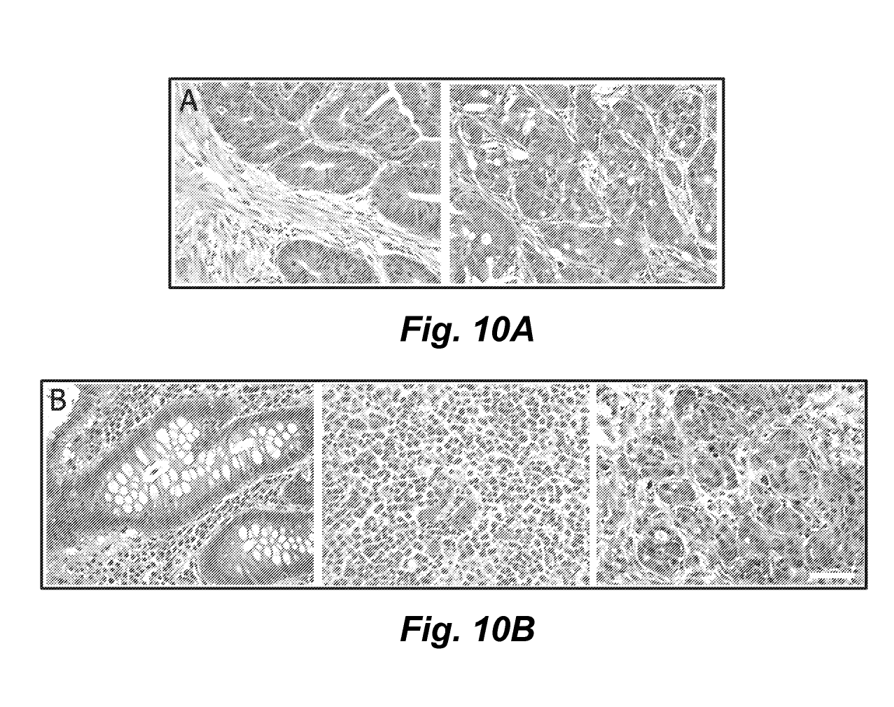

The xenograft model has been widely used for testing the tumorigenicity of putative cancerous cells such as colitis-derived cells, the results of which are shown in FIGS. 9A-9D. Thus, xenografting of ESA^{high}/ALDH1-positive primary tumor and colitic cells into NOD/SCID mice to determine tumor initiation and differentiation potential. Quantification of epithelial cells in grossly normal and colitic tissues supported the observations that ALDH1 is over-expressed in colitic epithelium (as shown in FIG. 9D). As few as 25 DAPI^{low}ESA^{high}ALDH1^{high} colonic cancer cells reproducibly generated serial xenografts with typical adenocarcinoma phenotypes, as shown in FIG. 10A.

To determine whether colitic tissue might bear the same tumorigenic potential as frank colon cancer, random tissue was isolated from patients presenting for resection of their colorectum due to chronic ulcerative colitis, with the majority (19/22) of these patients presenting for surgery due to medically refractory disease. Classically, ulcerative colitis may be more severe distally in the colon, and less severe or relatively normal in the proximal colon. Therefore, portions of the proximal colon were retained as a matching control tissue.

Proximal biopsies often revealed quiescent or nearly normal histology. For every specimen retrieved, the pathologic evaluation based on histology of the flanking sections to the distal implanted tissues showed only colitis without evidence of either dysplasia or adenocarcinoma. Colitis samples were implanted into the subcutaneous flank of NOD-SCID murine hosts, and were observed for up to 5 months.

In contrast to frank colorectal cancer, in which now routinely more than 75% (Dalerba et al., (2007) Supra) of the cancer tissue engraft successfully, xenografts evolved from only the three of twenty-two colitic specimens (13.6%). No tumors were found in the contralateral flank containing the proximal normal colonic tissue implants from the same patients. Similar to the frank colon cancer xenografts, all three of the primary xenografts resulting from the colitic tissues could form secondary xenografts, as shown in FIG. 10B, and were serially passaged in mice.

Secondary xenografts were formed from both bulk primary xenograft cells and flow cytometric sorted for DAPI^{low}ESA^{high}ALDH1^{high} cells. ESA was included to ensure isolation of predominantly epithelial cells rather than any potential ALDH1^{high} hematopoietic cells in the colon/xenograft. As few as 50 DAPI^{low}ESA^{high}ALDH1^{high} cells were capable of tumorigenesis via serial passage of the xenograft. Further, these tumors could be sequentially passaged at least ten passages with continued enrichment for ALDH 1^{high} cells.

The colitic cells within the early passage anaplastic xenografts did not stain for a series of tissue specific markers including CDX2, c-kit, CD3, CD2, β-catenin, CD30, vimentin, CK20, cam52, and CD45. While CDX2 was employed to evaluate differentiated intestinal tissues, both CD3 and CD2 (pan T-cell and T-cell subset) markers were absent. Similarly, the CD45, or common leukocyte antigen, was absent. CD30, a marker of anaplastic lymphomas, was not seen. Staining for neither CK20, a marker of differentiated colorectal carcinomas, nor CAM 5.2, a low molecular weight cytokeratins, was visualized. Further evaluation included S100, evident in some melanomas and therefore included in the differential for anaplastic lesions, was absent. To evaluate the possibility that these tumors were variants of gastrointestinal stromal tumors, they were stained with the CD117 antibody (c-kit antibody), and they were negative for this marker. Instead, they displayed a classic anaplastic morphology of expanded, homogeneous cells (FIG. 10B, middle).

Continued xenograft passage of ESA^{high}ALDH1^{high} selected cells resulted in progression of tumor morphology to a phenotype which was diagnostic of adenocarcinoma, with hallmark production of mucus and glandular formation (FIG. 10B, right). In those colitic tissues with successful engraftment, the latency of tumor growth from bulk cell populations was similar to cancer counterparts with onset of tumor growth averaging six weeks. Enrichment for ESA^{high}ALDH1^{high} selected cells resulted in palpable tumors in an average of two weeks. Once palpable, xenografts grew at similar rates regardless of source. Matched grossly normal tissues taken from the same patients did not engraft (N=22). This finding to categorizing the original xenograft initiating cells isolated from colitis patients as precursor-Colon Cancer Stem Cells or pCCSC in order to distinguish them from the CCSC isolated from frank colon cancer.

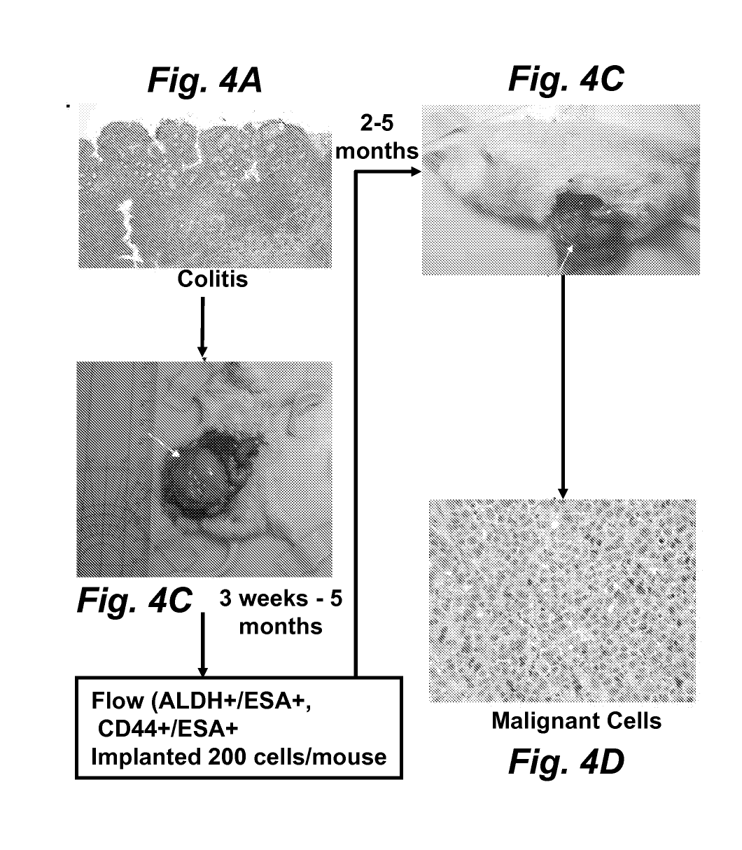

A colitic colon was removed at surgery and a 1 cm³ volume of tissue was retrieved from the inflammed region thereof. Prior to further processing, flanking sections were removed to validate the histology. As shown schematically in FIGS. 4A-4D, colon cancer initiating strategies, as described in Example 11, above, were used to engraft tumorigenic cells from colitis into a recipient animal. The resulting tumors were histologically anaplastic, and were able to engraft subsequent hosts with our selection criteria.

The anaplastic cells likely indicated that a primitive (stem) cell has been selected which has the capacity of self-renewal. (Note that using sporadic colon cancer, and directly sorting from the tumor, yields a phenotype which is less well-differentiated, yet is still clearly an adenocarcinoma, as shown in a comparison between FIGS. 2A and 2D). The patient characteristics are presented in Table 1, revealing that in three of eleven attempts, engraftment and tumorigenicity resulted from using from colitic tissue as the sorted and engrafted cells.

TABLE 1
Patient characteristics: indication for surgery,
histology of flanking sections, engraftment

| | Indication | Pathology of Flanking | Engraft- | Xenograft |
|---|---|---|---|---|
| Patient | for Surgery | Sections | ment | Pathology |
| 1 | Dysplasia/ | Atrophy, severe chronic | Yes | Anaplastic |
| | Colitis | colitis with lymphoid | | |
| | | hyperplasia, no dysplasia | | |
| 2 | Refractory | Intense active colitis with | Yes | Anaplastic |
| | colitis | lymphoid hyperplasia, no | | |
| | | dysplasia | | |
| 3 | Dysplasia/ | Villiform, intensely | No | NA |
| | Colitis | distorted, mildly active, | | |
| | | lymphoid hyperplasia, no | | |
| | | dysplasia | | |
| 4 | Refractory | Ulcer, severe chronic | No | NA |
| | Colitis | colitis in quiescent phase | | |
| 5 | Refractory | Intense active colitis with | No | NA |
| | Colitis | lymphoid hyperplasia, no | | |
| | | dysplasia | | |
| 6 | Refractory | Quiescent chronic colitis, | No | NA |
| | Colitis | no dysplasia | | |
| 7 | Refractory | Ulcer, chronic colitis, no | No | NA |
| | Colitis | dysplasia | | |
| 8 | Refractory | Quiescent colitis, adenoma | No | NA |
| | Colitis | | | |
| 9 | Refractory | Quiescent disease, minimal | Yes | Anaplastic |
| | Colitis | distortion, no dysplasia | | |
| 10 | Refractory | Quiescent disease, no | No | NA |
| | Colitis | dysplasia | | |
| 11 | Refractory | Quiescent disease, no | No | NA |
| | Colitis | dysplasia | | |

Furthermore, using CD44, ALDH1, and ESA, as markers of colon cancer CS/PC, rare cells from these xenografts were capable of perpetuating tumors. Examination of the flanking sections failed to reveal dysplasia or cancer. These results indicate that colitic epithelium, even in the absence of frank dysplasia or cancer, has the capacity to foster tumorigenic growth.

### Example 14

### Co-Culture of Cancer-Associated Fibroblasts with Epithelial Cells Increases Enhanced Proliferation

To determine the localization of fibroblasts relative to CS/PCs, immunofluorescence detection specific for ALDH1 was used as a putative CS/PC marker, and smooth muscle actin as a marker for fibroblasts. Fibroblasts were isolated from peritumoral and colitic tissues (see Bhowmick N. A, (2004) Nature 432: 332-337,). Next fibroblast isolates expression of smooth muscle actin was determined. Finally, a fibroblast-epithelial co-culture was formed, and this culture assayed for proliferation. Thus, dual immunofluorescence was used to determine the localization of the ALDH1 expressing crypt cells (FITC, green), while pericryptal fibroblasts were stained with smooth muscle actin (phycoerythrin, red). Cancer-associated fibroblasts and the HT29 colon cancer cell line were placed in co-culture and cell proliferation was assayed by measuring the incorporation of 5-bromo 2-deoxyuridine (BrdU) chemiluminescence

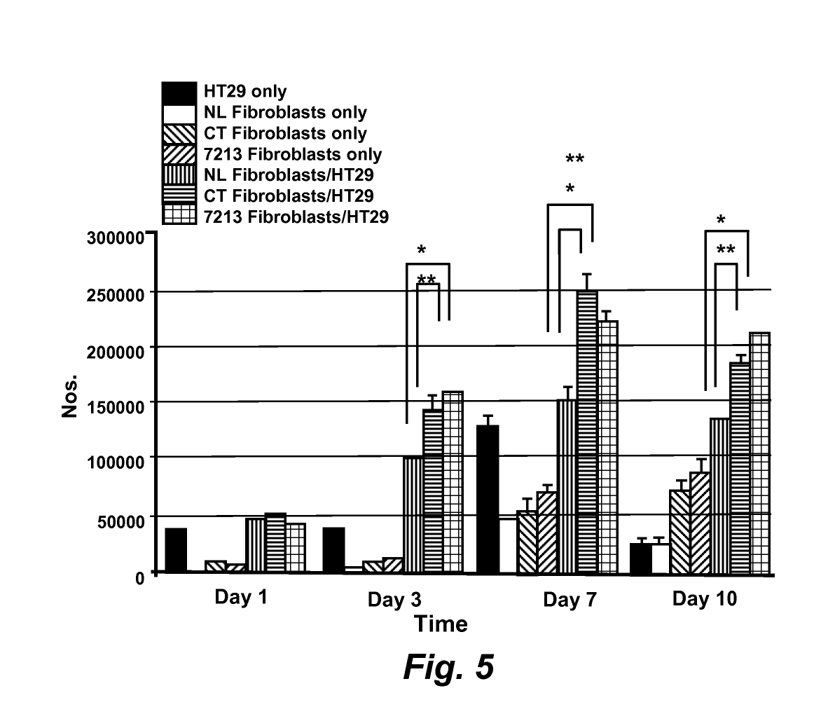

Immunofluorescence demonstrated that ALDH1-positive colonic crypt cells are at the base of the crypt adjacent to SMA-positive fibroblasts. Colon cancer-associated and colitis-associated fibroblasts may be isolated from primary tumors. Colon cancer-associated fibroblasts stimulate proliferation when co-cultured with the HT29 colon cancer cell line. Colitis-associated fibroblasts stimulate proliferation in co-culture with the HT29 colon cancer cell line. As shown in FIG. 5, the degree of proliferation was intermediate between the fibroblasts isolated from grossly normal colon (“NL”) and the proliferation seen when placed in co-culture with the colon cancer fibroblast cell line (“7213”).

### Example 15

### Epithelial Marker Expression of Primary Tumors, Xenografts, and Spheres

Neural stem cells can be characterized by their ability to form sphere shaped clusters, termed “neurospheres”, in serum free medium (Reynolds & Weiss, (1992) Science 255: 1707-1710; Reynolds & Rietze, (2005) Nat. Methods 2: 333-336). Sphere formation has subsequently been used to isolate both normal and cancer stem/progenitor cells from a wide variety of tissues including the colon (Ricci-Vitiani et al., (2007) Nature 445: 111-115; Todaro et al., (2007) Cell Stem Cell 1: 389-402; Todaro et al., (2008) Cell Cycle 7: 309-313). The data of the present disclosure established conditions that support the formation of colon spheres from both frank colon cancer and colitis, but not from normal colon.

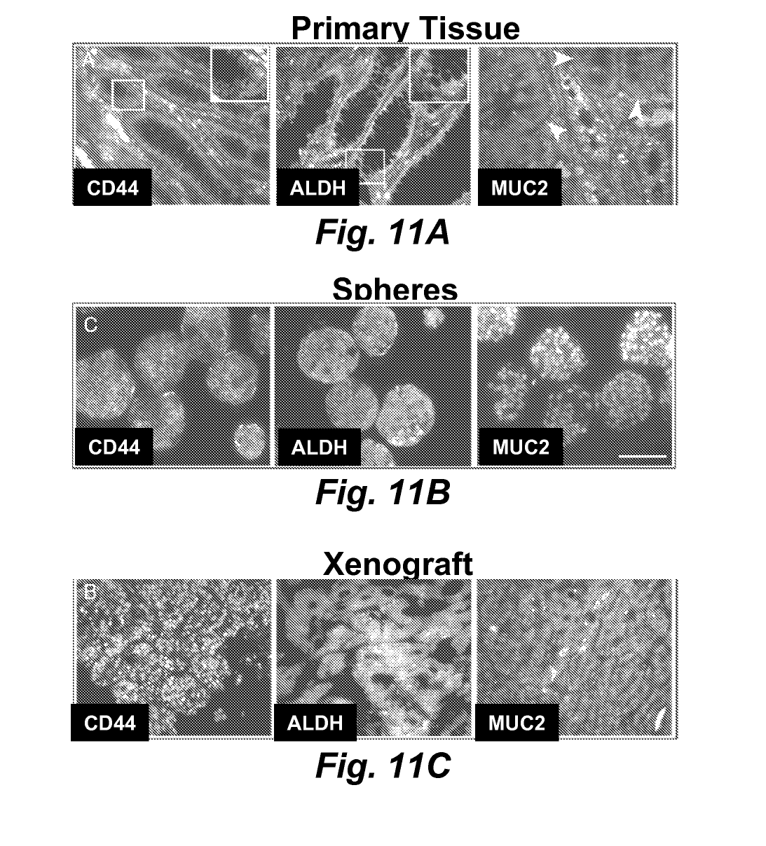

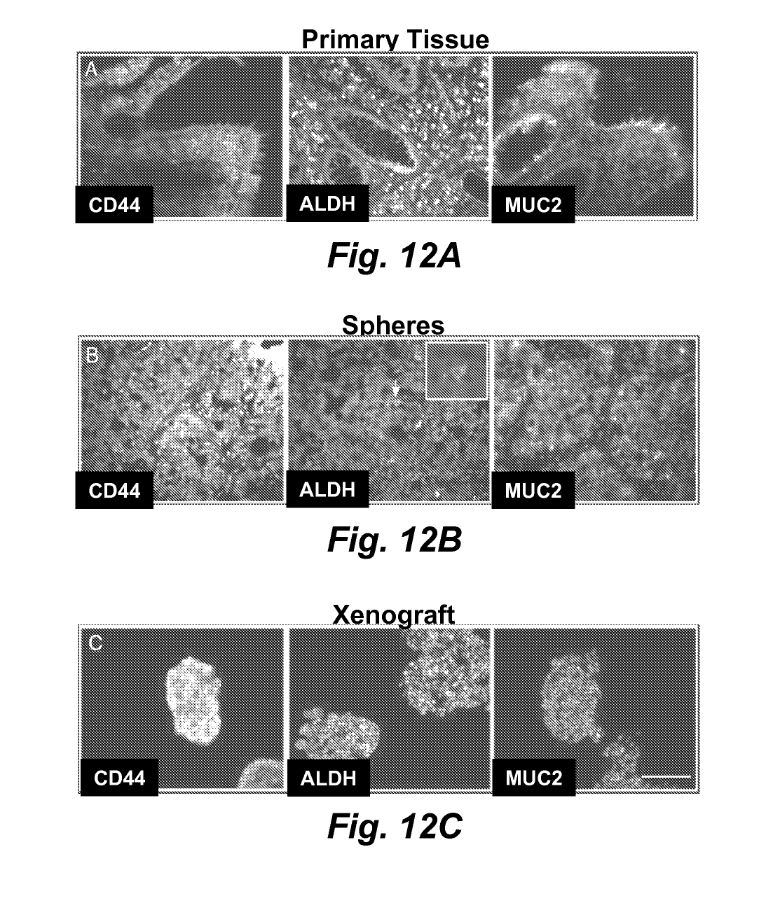

Immunohistochemistry was used to delineate the phenotypes of the original tissues compared with both xenografted tissues and with the colon spheres, as shown in FIGS. 11 and 11. MUC2 is present in goblet cells during mucin formation, and is indicative of epithelial differentiation (Blache et al., (2004) J. Cell Biol. 166: 37-47). Here, MUC2 staining was observed in original tissues and in the xenografts from both colon cancer and serially passaged colitis sources with an adenocarcinoma histological profile (see FIGS. 11, and 12A and 12B, right panels). Expression of MUC2 was only visualized at the outer edges of the spheroid colonies generated from the frankly malignant source and not in the colitis spheres (FIGS. 11 and 12C, right panels), reflecting a more anaplastic nature.

ALDH1 and CD44 immunoreactivities were also compared revealing dispersed cytological localization in both the primary tumors and resulting xenografts. CD44 expression was seen in primary tissues, xenografted tissues, and spheres from both origins (FIGS. 12 and 11A-11C, left panels). However, though ALDH1 expression was seen in sections of all primary patient samples, the expression was much more restricted in successful xenografts and in the spheres from colitic origins (FIGS. 12B and 11C middle panels).

### Interleukin 8 is Secreted by Colitic and Colon Cancer Stromal Fibroblasts

Multiple pathways are involved in tumorigenic growth. To define possible etiologies contributory to the proliferation discussed in Example 14, above, and summarized in FIG. 5 (co-culture of cancer-associated fibroblasts (“7213”), fibroblasts from grossly normal colon in colitis (“NL”), and colitis-associated fibroblasts (“CT”) with the epithelial colon cancer cell line (HT29) revealed increased proliferation on days 3, 7 and 10. **, p<0.05; *, p<0.01, N=2), the cytokines exhibiting increased secretion from the fibroblasts isolated from colitic and colon cancer-associated fibroblasts were investigated.

Fibroblasts were isolated from grossly normal, colitic, and malignant colon tissue. They were confirmed both by histological appearance and by staining with smooth muscle actin. These cells were plated at 1×10⁵ per well in 24-well plates. The cells were permitted to adhere overnight, and the serum-containing media was removed, washed with PBS, and changed to serum-free media. Cytokine arrays (Chemicon, Panomics) were for angiogenesis and cytokines/growth factors. The cellular supernatants (conditioned media) were assayed per manufacturers' instructions.

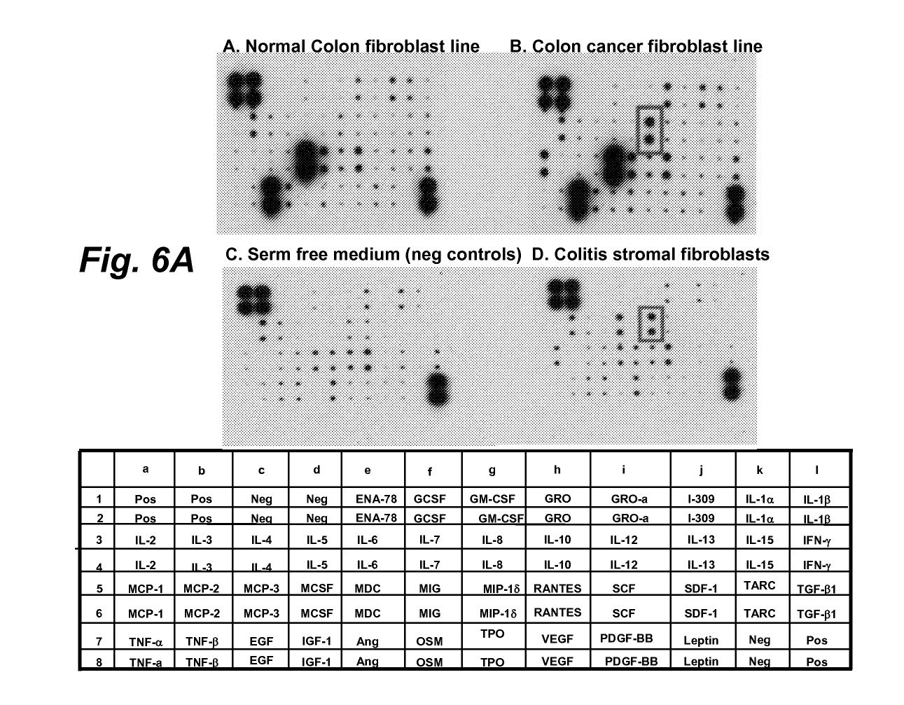

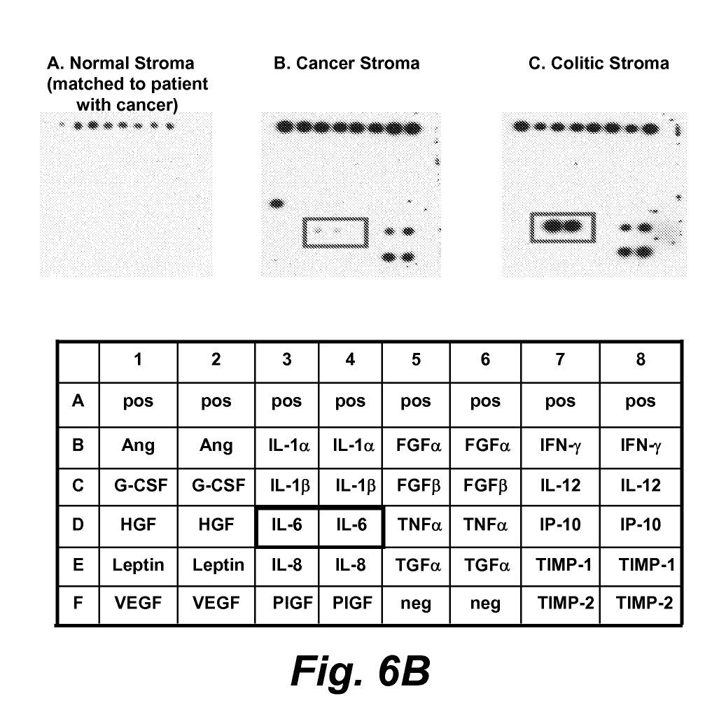

Fibroblast supernatants were successfully assayed for cytokines and growth factors. In the cytokine arrays, as shown in FIGS. 6A and 6B, notable positives were noted as follows: (in FIG. 6A, panel B): Colon cancer fibroblast cell line (versus the normal colon fibroblast cell line shown in FIG. 6A, panel A): IL-8, MCP-1, and Ma; (in FIG. 6A, panel D): Colitis-associated fibroblasts (versus the normal colon fibroblast cell line shown in FIG. 6A, panel A): IL-8, IL-6.

In the angiogenesis array (FIG. 6B, panels A-C), IL-8 and HGF were notably positive for the colon cancer stroma versus the matched normal stroma. The colitis-associated stroma also secreted IL-8. In contrast to the stroma from normal colon, both the fibroblasts from colon cancer and colitis secreted IL-8.

### Example 16

### The Receptor for Interleukin 8 is Expressed by the Tumorigenic Epithelium in the Xenografts

To determine whether IL-8 might be contributing to tumorigenicity from both colon cancer, and colitis, the presence or absence of the receptor for IL-8 in successful xenografts was investigated.

Immunohistochemistry on tumorigenic xenografts for IL-8 receptor, CXCR1/2, with the chromogen AEC (red) was used and showed that the IL-8-receptor, CXCR1/2, was expressed on successful tumorigenic xenografts from either colon cancer or colitis.

### Example 17

### Spheroid Cultures Derived from Both ALDH1^{high}-Sorted Colon Cancer or -Colitis Xenografts can be Maintained in Culture, and are Conducive to Lentiviral Transduction

To determine whether the markers CD44 and ALDH1 would allow for enrichment for cells with the capacity to form spheroid cultures, colon cancer and colitis cells from disrupted tissues were sorted to evaluate whether maintenance in vitro with sequential expansion and lentiviral transduction was possible.

Flow cytometry was used against the ALDH1 and CD44 markers, selecting for those cells from colon cancer xenografts that were human (H2K^{d-low}) and ESA^{high}. The resulting populations were plated at 2−10×10³ cells/well in a 6-well, low attachment plate (Corning) in DMEM/F12 media with N2 additives (Invitrogen) and growth factors (considered as low-attachment conditions in serum-free media).

When multiple spheres greater than 100 microns in size were visualized, they were removed, trypsinized, and expanded into subsequent cultures. For lentiviral transduction, 10³ individual cells from these spheres were isolated and transduced with a lentiviral construct containing CMV promotor-driven dsRed.

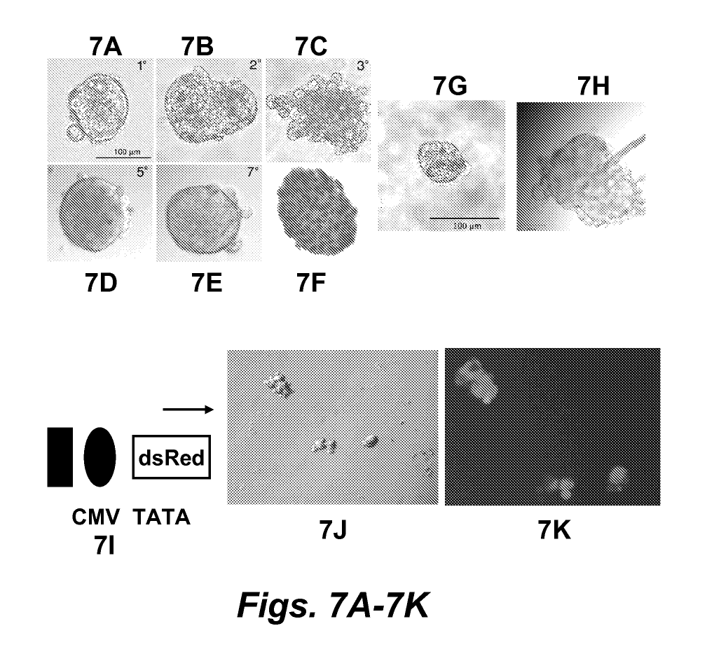

Colon cancer initiating cells were enriched by sorting for CD44 and ALDH1. Notably, spheroid cultures were isolated from CD44 cells, as shown in FIGS. 7A-7F). Both ALDH1 enriched colon cancer and colitis epithelium were able to be maintained in vitro as spheres (shown in FIGS. 7G and 7H, respectively). Isolates that were ALDH1^{low} or CD44^{low} failed to sustain growth in culture, and thus were unable to be propagated. Lentiviral transduction with a reporter for dsRed was possible (see FIGS. 7I-7K), supporting this mode of transduction as a suitable method for the introduction of genetic material into the isolated colon and colitic stem cells of the present disclosure.

### Example 18

### Bioimaging of Human Colon Cancer Cells into Immuno-Compromised Mice

The orthotopic implantation of colonic xenografts requires a laparotomy, and subsequent serial examination would require sacrifice of the animal. Techniques which enable the noninvasive, sequential assessment of engraftment, with sequelae including growth and metastases without sacrifice of the animal would permit elucidation of tumor biology. Thus the number of animals required for experimentation would be minimized.

To determine the feasibility of this technology, the HT29 colon cancer cell line was used to determine whether orthotopic (cecal) tumor establishment was possible. This cell line was stably infected with the luciferase gene via lentivirus transfection. Once bioluminescence imaging was conducted, digital grayscale animal imaging was acquired followed by acquisition and overlay of a pseudocolor image representing the spatial distribution of detected photons emerging from active luciferase within the animal. Signal intensity was quantified as the sum of all detected photons within the region of interest per second.

The orthotopically-placed luciferase lentivirus transfected cells were identified using bioimaging. Bioimaging was able to identify a non-visible focus of metastasis confirmed by microscopy with H&E staining.

Murine xenografts may be used to determine in vivo sequelae. The exploitation of bioimaging allows for detection of non-visible foci and for sequential noninvasive examination.

### Example 19

### Stromal Microenvironment Changes in Colitis and Cancer

Colonic stromal cells were isolated from the colitic milieu and compared the cytokine profile of conditioned medium from the colitic fibroblasts to normal colon and malignant colon fibroblasts using cytokine array analysis. The simultaneous injection of cancer-associated stroma with epithelial cells was tested as a combination that could decrease the tumor latency period, thereby advancing the appearance rate of a xenografted tumor. Colon cancer associated fibroblasts were co-injected with colon cancer-derived dissociated spheres in a 1.1 ratio (10³ of each type of cell) into the subcutaneous flank of a NOD-SCID mouse. The opposing flank of the animal received a control injection of 2×10³ fibroblasts or dissociated spheres, but not both together.

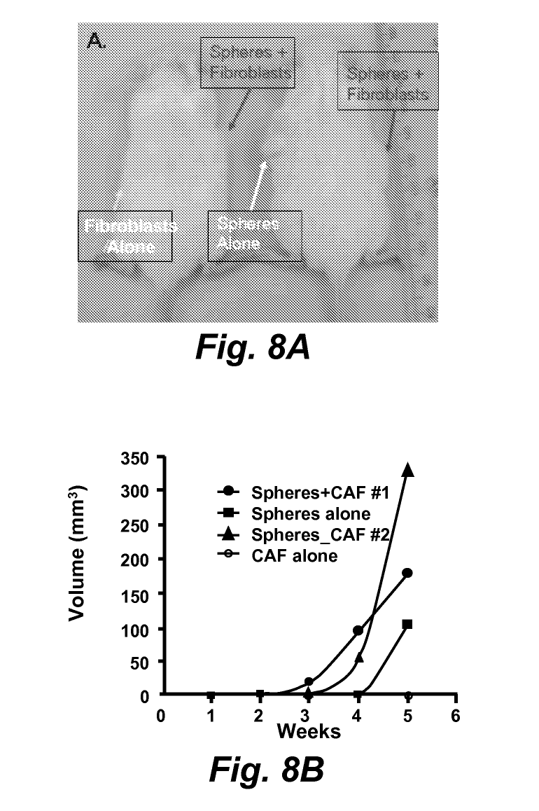

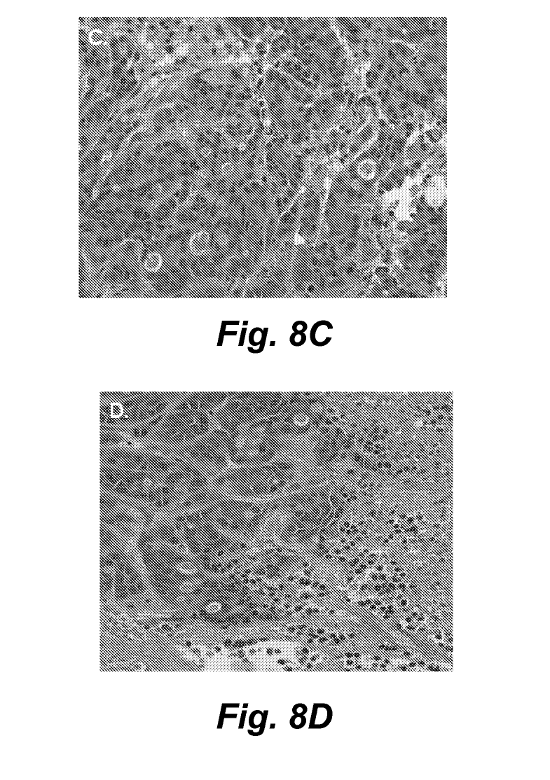

The co-injection of colon cancer-associated fibroblasts with colon cancer-derived spheres resulted in tumors in the subcutaneous tissues of NOD-SCID mice, as shown in FIG. 8A. The injections exhibited decreased tumor latency compared to the injection of spheres alone, as shown in FIG. 8B. The resulting histology showed nests of adenocarcinoma (FIGS. 8C and 8D).

The addition of cancer associated fibroblasts, therefore, decreased tumor latency and the resulting histological phenotype was predominantly that of the epithelial component.

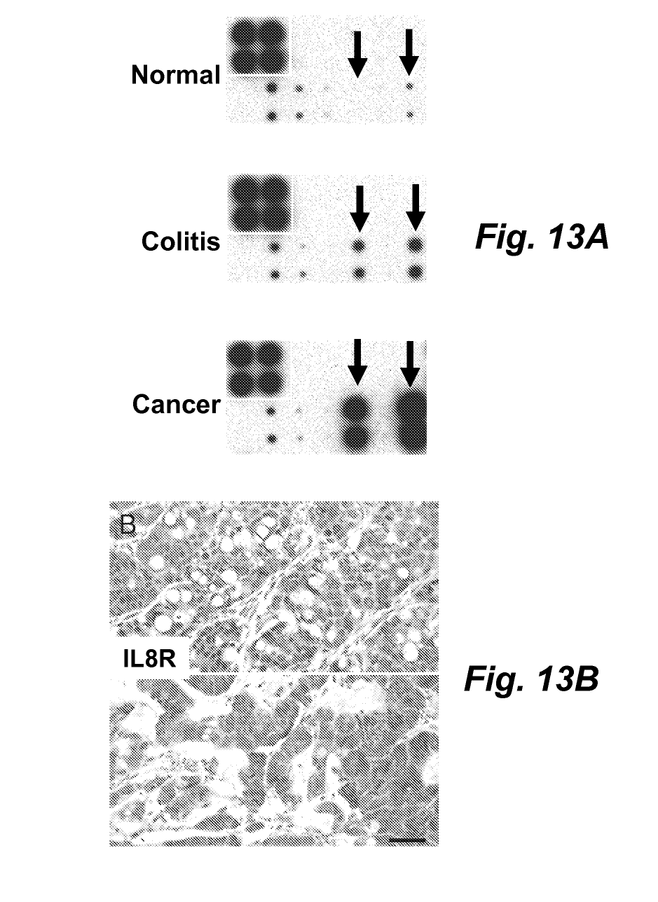

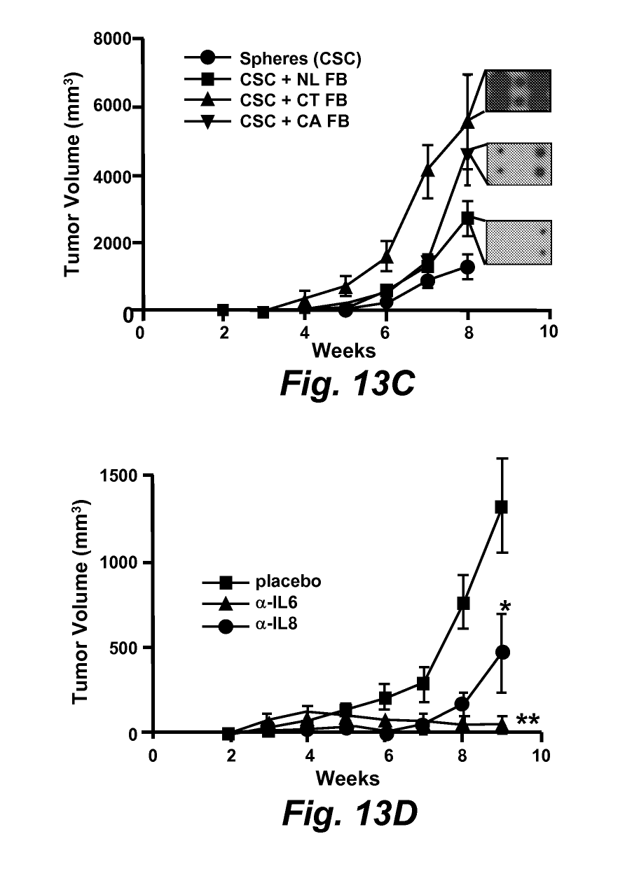

The expression of IL-6 and IL-8 was low to undetectable from the normal stromal cell medium as shown in FIG. 13A, top, but was substantially higher in medium taken from both the colitic (FIG. 13A, middle) and frankly malignant (FIG. 13A, bottom) stromal environments. In most cases the colitic stroma produced an intermediary amount of IL-6 and IL-8 compared to the baseline expression of normal colon stroma and high-level expression from colon cancer stroma. IL-8 has at least one high affinity receptor, CXCR1/2, that is prominently expressed in malignant colon and colitic xenografts of ESA^{high}ALDH1^{high} sorted cells, as shown in FIG. 13B).

It was next evaluated whether colitis or cancer derived stroma enhance the ability of colon cancer spheres to form xenografts. Sphere-derived single cell suspensions were injected alone, or in the presence of normal colon/colon cancer/colitis-associated fibroblasts, and the recipient mice monitored for tumor volumes and latencies. Those mice with both sphere-derived cells and cancer-associated fibroblasts developed tumors with a markedly decreased latency—mean time to a palpable tumor of about 2 weeks with cancer/colitis fibroblasts versus about 6 weeks for spheres alone, or with normal fibroblasts (FIG. 13C) providing evidence that fibroblast-derived IL-6 and/or IL-8 plays a role in colon tumorigenesis. Moreover, colon cancer stem cells, when transplanted alone, result in longer tumor latency and smallest tumor volume in xenograft experiments (ANOVA, Duncan's multiple comparison p<0.05). Mixed model analysis for tumor growth rate in the colon cancer sphere with colitic fibroblasts revealed a growth rate that was significantly higher than that in the colon cancer spheres co-injected with either the colon cancer fibroblasts, normal colon fibroblasts, or spheres alone (p=0.0006, <0.0001, and <0.0001, respectively).

The tumor growth rate for the colon cancer spheres with colon cancer fibroblasts group was significantly higher that that in colon cancer spheres alone group (p=0.0006). Injections consisting of fibroblasts alone failed to generate a xenograft, which was confirmed at necropsy. As shown in the insets of FIG. 13C, for this experiment, the colitic fibroblasts secreted more IL-6 and IL-8 than the colon cancer fibroblast cell line.

To further determine a role for these cytokines in tumorigenesis, slow-release pellets containing either anti-IL-8 or anti-IL-6 antibodies were implanted adjacent to the xenografted cells. Both conditions resulted in significantly reduced tumor volume compared to the control group (FIG. 13D). Moreover, palpable tumors increased in size after 8 weeks, the period at which the antibody supply in the pellets was exhausted. Together, these data indicate a direct role for these cytokines in tumor growth in vivo.

### Example 20

### Colon Cancer Derived Spheres Yield Adherent Cultures when Placed in Serum-Containing Media, and Express MUC2, Resembling Mature Crypts

Colon cancer-derived spheroid cultures were established. Spheres were harvested and placed in differentiating conditions with serum-containing media in tissue culture treated plates. A fraction of these cultures were then removed and replaced in sphere-conducive conditions. Immunofluorescence for mature epithelial markers was performed.

Spheroid cultures were maintained in the absence of serum in low attachment conditions. However, when placed in routine tissue culture plates with 1% serum, adherent colonies with morphology resembling colon cancer cell lines was observed. When the adherent cultures were removed and placed in low-attachment conditions without serum, they again adopted the spheroid morphology.

Well-differentiated normal colon crypts reveal classical staining for the markers CD44, CDX2, CK19, and MUC2. Immunofluorescence for the differentiated phenotype as defined by MUC2 was found to be present in a fraction of the culture. Thus, in a fraction of the spheroid cultures, differentiating conditions produced adherent cultures that resembled commercially-available colon cancer cell lines. Importantly, when replaced in sphere-conducive settings, spheres were readily evident. Further, MUC2, a marker of mature epithelium, was present in the adherent cultures. However, Lgr5 (Barker, Nature, 2007), a putative marker of normal colon epithelial stem cells in mice, was absent.

Thus, spheroid cultures derived from the culturing of isolated colon cancer CS/PCs exhibit phenotypic plasticity in vitro, thereby providing a tool for the dissection of both colon cancer pathogenesis, and the influence of the microenvironment on the colon cancer CS/PC.

### Example 21

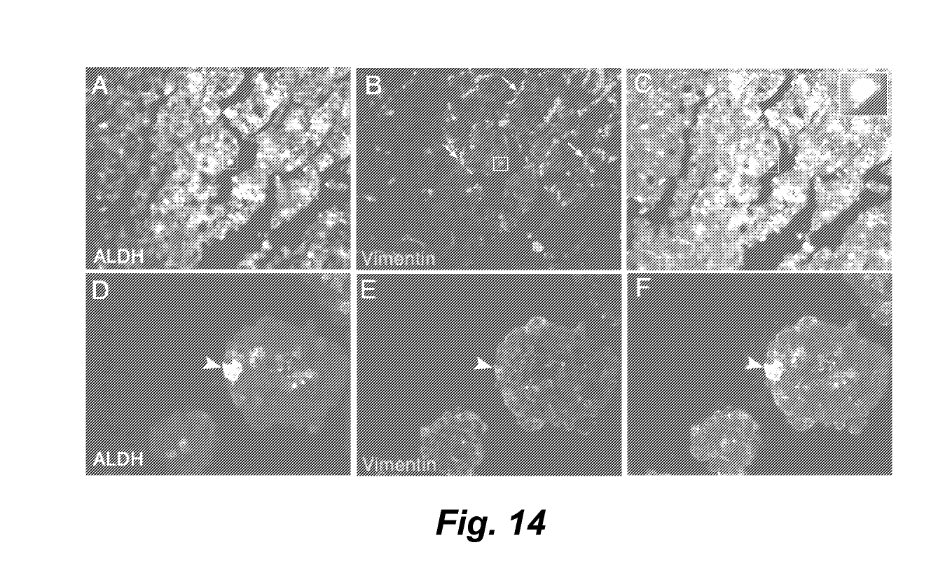

As shown in FIG. 14, panels A-F, colitis xenografts displayed immunofluorescence for ALDH1 (FITC, A), and vimentin (PE, B), with merged images shown in panel C demonstrating the localization of the putative stem cell marker, aldehyde dehydrogenase. In rare instances, vimentin, a mesenchymal marker associated with EMT, co-localized in the epithelial tissue, indicative of an EMT event within the tumor. Most of the vimentin expression was limited to areas between the epithelial glandular structures (white arrows). However, in rare instances, and similar to the results shown in Panels A-C, ALDH1 and vimentin exhibited co-localization of expression in rare cells within the glandular structures (arrows).

In FIG. 14, panels D-F are shown colitis spheroid colonies. The sphere cultures displayed similar patterns of vimentin/ALDH1 co-expression (white arrowheads), demonstrating that the sphere cultures represented a system for early diagnosis of tumorigenesis from inflammatory (colitic) tissue.

1-50. (canceled)

51. An isolated mammalian pluripotent colon epithelial stem/progenitor cell (CS/PC) having the detectable marker aldehyde dehydrogenase 1 (ALDH1), or a population of said cells, substantially free of cells that do not have the detectable ALDH1 marker, and wherein each CS/PC is characterized as: (i) further comprising at least one detectable marker selected from the group consisting of: ESA, c-kit, Oct 3/4, Nanog, activated Stat-3, CXCR4, CXCR1/2, CD133, SCA-1, Tra-1-60, CD44, CD73, CD90, CD105, vimentin, and Stro-1; and (ii) when isolated from a colitic colon and engrafted into a recipient animal forms an anaplastic mass of cells that when serially passaged from the anaplastic mass into a recipient animal or series of animals, forms an adenocarcinoma comprising cells having the characteristics of a colon cancer cell.

52. The population of isolated mammalian pluripotent colon epithelial stem/progenitor cell (CS/PC) having the detectable marker aldehyde dehydrogenase 1 (ALDH1) according to claim 51, wherein the population of CS/PCs is cultured under in vitro conditions, thereby forming a cell-based spheroid.

53. The isolated mammalian pluripotent colon epithelial stem/progenitor cell (CS/PC), or population of said cells according to claim 51, wherein said cell or population of cells is co-cultured with a population of isolated mammalian colonic stromal fibroblast cells.

54. The isolated mammalian pluripotent colon epithelial stem/progenitor cell (CS/PC), or population of said cells according to claim 51, wherein the co-culture is cultured in a recipient subject animal.

55. A method of isolating a mammalian pluripotent colon epithelial stem/progenitor cell (CS/PC), or a population of said cells, from an animal or human colon, wherein each CS/PC is characterized as having the detectable marker aldehyde dehydrogenase 1 (ALDH1) and at least one detectable marker selected from the group consisting of: ESA, c-kit, Oct 3/4, Nanog, activated Stat-3, CXCR4, CXCR1/2, CD133, SCA-1, Tra-1-60, CD44, CD73, CD90, CD105, and Stro-1, said method comprising the steps of:
(i) obtaining a tissue sample from the colon of an animal or human subject;
(ii) disrupting the tissue sample, thereby obtaining a cell suspension, wherein the cell suspension is substantially comprised of single cells;
(iii) contacting the cell suspension with at least one ligand species capable of selectively binding to a cell marker or combination of markers selectively identifying the mammalian pluripotent colon epithelial stem/progenitor cell (CS/PC), and wherein each ligand species comprises a detectable label;
(iv) identifying a cell or population of cells binding to the ligand species or combination of ligand species; and
(v) isolating the identified cell or population of cells selectively binding to the ligand species or combination of ligand species from a population of cells not selectively binding to the ligand species or combination of ligand species, thereby obtaining an isolated population of mammalian pluripotent colon epithelial stem/progenitor (CS/PC) cells.

56. The method according to claim 55, further comprising the step of:
culturing the isolated population of mammalian pluripotent colon epithelial stem/progenitor (CS/PC) cells in a medium under proliferative conditions, wherein the cells are allowed to form a spheroid population of cells (colonspheres), or an adherent layer of cells.

57. The method according to claim 56, wherein the medium comprises a population of isolated colon stromal fibroblasts.

58. The method according to claim 56, wherein the medium comprises at least one cell separation compound selected from the group consisting of: a chemical-separating compound, a physical-separating compound, and an anti-adhesive compound.

59. The method according to claim 55, wherein the tissue sample is from a colitic colon, and wherein the isolated CS/PCs, when engrafted into a recipient animal, forms an anaplastic mass of cells that when serially passaged from the anaplastic mass into a recipient animal or series of animals, forms an adenocarcinoma comprising cells having the characteristics of a colon cancer cell.

60. The method according to claim 56, further comprising the steps of:
culturing colonspheres;
dissociating the colonspheres into single cells;
culturing said single cells to near confluence and harvesting;
reseeding harvested cells into suspension (non-adherent) cultures;
identifying self-renewing cells by formation of secondary spheres; and
isolating self-renewing pluripotent CS/PC clones therefrom, wherein the self-renewing pluripotent CS/PC clones express ALDH1 and at least one of the group consisting of: ESA, c-kit, Muc1, Muc2, CK19, Oct 3/4, Nanog, activated Stat-3, CXCR4, CXCR1/2, CD133, SCA-1, Tra-1-60, CD44, CD73, CD90, CD105, vimentin, and Stro-1.

61. A method of determining the prognosis for a patient for developing a colon cancer, the method comprising the steps of:
(i) obtaining an isolated tissue sample from the colon of an animal or human patient;
(ii) obtaining a tissue section from the isolated tissue section; and
(iii) detecting the presence of at least one marker in the tissue section, wherein the marker or plurality of markers indicate the presence of a pluripotent colon epithelial stem/progenitor cell (CS/PC) wherein each CS/PC is characterized as having the detectable marker aldehyde dehydrogenase 1 (ALDH1) and at least one detectable marker selected from the group consisting of: ESA, c-kit, Oct 3/4, Nanog, activated Stat-3, CXCR4, CXCR1/2, CD133, SCA-1, Tra-1-60, CD44, CD73, CD90, CD105, vimentin, and Stro-1, thereby indicating the prognosis of the patient for developing a colon cancer.

62. The method according to claim 61, wherein the plurality of markers comprises ALDH1 and at least one marker selected from the group consisting of: ESA, CD133, CD44, and vimentin.

63. The method according to claim 61, further comprising the steps of:
disrupting the tissue sample, thereby obtaining a cell suspension, wherein the cell suspension is substantially comprised of single cells;
contacting the disrupted tissue sample with at least one labeled ligand species capable of selectively binding to a cell marker indicative of a mammalian pluripotent colon epithelial stem/progenitor cell (CS/PC);
isolating a pluripotent colon epithelial stem/progenitor (CS/PC) cell or a population of said cells from the disrupted tissue sample by isolating a labeled ligand species bound to a cell or cells of the disrupted tissue sample, thereby obtaining an isolated population of mammalian pluripotent colon epithelial stem/progenitor (CS/PC) cells;
culturing the isolated population of pluripotent colon epithelial stem/progenitor (CS/PC) cells in a medium under conditions favorable for the proliferation of pluripotent colon epithelial stem/progenitor (CS/PC) cells; and
determining whether the cultured cells comprise colon epithelial stem/progenitor (CS/PC) cells; thereby determining the prognosis for the patient developing a colonic cancer.

64. The method according to claim 61, wherein the tissue sample is isolated from a patient having the symptoms of a colitis, and wherein the CS/PCs, when isolated from the colitic colon, when engrafted into a recipient animal, forms an anaplastic mass, and wherein, when said cells are serially passaged from the anaplastic mass into a recipient animal or series of animals, forms an adenocarcinoma, wherein the cells of the adenocarcinoma have the characteristics of a colon cancer cell.

65. The method according to claim 61, further comprising the step of:
culturing the cell suspension in a medium under conditions wherein the cells are allowed to proliferate to form a spheroid population of cells; and
identifying the cells of the spheroid as colon epithelial stem/progenitor (CS/PC) cells; thereby determining prognosis for the patient developing a colonic cancer; or, optionally
administering the isolated cells to a recipient animal and allowing the animal to develop a tumor; and
identifying the cells of the developed tumor as colon epithelial stem/progenitor (CS/PC) cells; thereby determining prognosis for the patient developing a colonic cancer.

66. The method according to claim 61, further comprising the step of:
co-culturing the cell suspension with colonic stromal fibroblasts isolated from the patient.

67. The method according to claim 61, further comprising determining the presence of an epithelial-mesenchymal transition event in the spheroid, wherein the presence of the epithelial-mesenchymal transition event indicates that at least one of the spheroid cells is metastatic; or determining the presence of an epithelial-mesenchymal transition event in the tumor, wherein the presence of the epithelial-mesenchymal transition event indicates that at least one of the spheroid cells is metastatic.

68. A kit comprising a container comprising a detectable ligand, or a combination of detectable ligands, wherein each species of detectable ligand specifically binds to an individual marker, wherein a marker, alone or in combination with at least one other marker, identifies a colonic stem/progenitor cell from a population of cells of a mammal, and wherein the at least one detectable ligand is ALDH1 and at least one marker selected from the group consisting of: ESA, c-kit, Muc1, Muc2, CK19, Oct 3/4, Nanog, activated Stat-3, CXCR4, CXCR1/2, CD133, SCA-1, Tra-1-60, CD44, CD73, CD90, CD105, vimentin, and Stro-1, and instructions for the use of the detectable ligand or combination of markers to identify a colonic stem/progenitor cell from a population of cells of a mammal.

69. A method of identifying a candidate therapeutic compound effective in reducing the proliferative status of a colonic stem/progenitor cell of a mammal, comprising the steps of:
culturing a population, or plurality of populations, of isolated CS/PCs, with a plurality of candidate therapeutic agents, wherein each population of CS/PCs expresses at least one marker selected from the group consisting of: ALDH1, ESA Oct 3/4, Nanog, activated Stat-3, CXCR4, CXCR1/2, CD133, SCA-1, Tra-1-60, CD44, CD73, CD90, CD105, vimentin, and Stro-1; and
identifying a candidate therapeutic agent that reduces the proliferation status of a culture of a population of CS/PCs.

70. A method of modulating the proliferative status of a colonic cancer cell comprising:
contacting a colonic cell with an effective amount of an agent, wherein the agent selectively binds to a cell surface marker, and wherein the agent when bound to the cell surface marker inhibits binding of a factor to the cell, thereby modulating the proliferative status of the colonic cell.
